(19) United States
(12) Patent Application Publication
(10) Pub. No.: US 2015/0253305 A1
(43) Pub. Date: Sep. 10, 2015

(54) METHODS OF IDENTIFYING COMPOUNDS FOR TREATING DEPRESSION AND OTHER RELATED DISEASES

(71) Applicant: NORTHWESTERN UNIVERSITY, Evanston, IL (US)

(72) Inventor: Joseph R. Moskal, Evanston, IL (US)

(21) Appl. No.: 14/434,911

(22) PCT Filed: Oct. 11, 2013
(86) PCT No.: PCT/US13/64625

§ 371 Date: Apr. 10, 2015

Related U.S. Application Data
(60) Provisional application No. 61/713,085, filed on Oct. 12, 2012.
(60) Provisional application No. 61/824,667, filed on May 17, 2013.

(51) Int. Cl. G01N 33/50; G01N 33/68

(52) U.S. Cl. CPC G01N 33/5058; G01N 33/6872; G01N 2333/82; G01N 2800/304

(57) ABSTRACT

The present disclosure relates in part to methods of identifying a candidate compound suitable for treatment of depression. In some embodiments, a candidate compound may be a NMDAR partial agonist.

### CROSS REFERENCE TO RELATED APPLICATIONS

This application claims the benefit of and priority to U.S. Provisional Patent Application Ser. No. 61/824,667, filed May 17, 2013, and U.S. Provisional Patent Application Ser. No. 61/713,085, filed Oct. 12, 2012, each of which is hereby incorporated by reference in its entirety.

### BACKGROUND

The N-methyl-D-aspartate (NMDA) receptor (NMDAR) has been implicated in neurodegenerative disorders including stroke-related brain cell death, convulsive disorders, and learning and memory. NMDAR also plays a central role in modulating normal synaptic transmission, synaptic plasticity, and excitotoxicity in the central nervous system. The NMDAR is further involved in Long-term potentiation (LTP).

The NMDAR is activated by the binding of NMDA, glutamate (Glu), and aspartate (Asp). It is competitively antagonized by D-2-amino-5-phosphonovalerate (D-APS; D-APV), and non-competitively antagonized by phenylcyclidine (PCP), and MK-801. Most interestingly, the NMDAR is co-activated by glycine (Gly) (Kozikowski et al., 1990, Journal of Medicinal Chemistry 33:1561-1571). The binding of glycine occurs at an allosteric regulatory site on the NMDAR complex, and this increases both the duration of channel open time, and the frequency of the opening of the NMDAR channel.

Recent human clinical studies have identified NMDAR as a novel target of high interest for treatment of depression. These studies conducted using known NMDAR antagonists CPC-101,606 and ketamine have shown significant reductions in the Hamilton Depression Rating Score in patients suffering with refractory depression. Although, the efficacy was significant, but the side effects of using these NDMAR antagonists were severe.

NMDA-modulating small molecule agonist and antagonist compounds have been developed for potential therapeutic use. However, many of these are associated with very narrow therapeutic indices and undesirable side effects including hallucinations, ataxia, irrational behavior, and significant toxicity, all of which limit their effectiveness and/or safety. Further, 50% or more of patients with depression do not experience an adequate therapeutic response to known administered drugs. There currently is no single effective treatment for depression, anxiety, and other related diseases.

Thus, there remains a need for improved treatments of depression, anxiety and/or other related diseases with compounds that provide increased efficacy and reduced undesirable side effects.

### SUMMARY

The present disclosure relates in part to methods of identifying a candidate compound suitable for treatment of depression. In some embodiments, a candidate compound may be a NMDAR partial agonist.

In one aspect, a method for identifying a candidate compound suitable for treatment of depression is provided. The method comprises exposing a cell to a potential compound in a culture medium, or administering a potential compound to an animal; retrieving a sample from the cell and/or culture medium, or from brain or neural tissue of the animal, at one or more predetermined time points; analyzing the sample for increased or decreased expression levels of Wnt1 and/or identifying the candidate compound as suitable for treatment of depression based on the increased expression level of Wnt1.

In another aspect, a method for identifying a candidate compound suitable for treatment of depression is provided. The method comprises exposing a cell to a potential compound in a culture medium, or administering a potential compound to an animal; retrieving a sample from the cell and/or culture medium, or from brain or neural tissue of the animal, at one or more predetermined time points; analyzing the sample for increased expression levels of at least one of the genes listed in Table 1 or 2 indicated with a G, or decreased expression levels or at least one of the genes listed in Table 1 or 2 indicated with a K, and identifying the compound as suitable for treatment of depression based on the increased expression level or decreased expression level.

In some embodiments, the sample has a gene expression pattern as provided in Table 1 or 2 with the indication “G” and the identifying is based on increased expression of those genes.

In some embodiments, a contemplated method further comprises analyzing the candidate compound for NDMA subunit NR2B synaptic plasticity.

In another aspect, a method for identifying a compound suitable for treatment of depression is provided. The method comprises exposing a cell to a potential compound in a culture medium, or administering a potential compound to an animal; retrieving a sample from the cell and/or culture medium, or from brain or neural tissue of the animal, at one or more predetermined time points; analyzing the sample for NMDA receptor NR2B subunit plasticity, and identifying the compound as suitable for treatment of depression based on inducing the NR2B plasticity.

In some embodiments, a candidate compound suitable for treating depression significantly induces NR2B dependent synaptic plasticity as compared to ketamine.

In some embodiments, the tissue is medial prefrontal cortex.

In some embodiments, the animal is a rodent or human, and the cell is a human or rodent cell.

In some embodiments, the compound modulates the NMDA receptor.

In some embodiments, the compound suitable for treating depression has fewer side effects as compared to ketamine.

In some embodiments, the compound does not have substantial addictive sensory motor grating and/or sedative effect.

In some embodiments, the cell is a eukaryotic cell.

In some embodiments, a contemplated method further comprises selecting the candidate compound from a library of compounds.

In yet another aspect, a method of identifying a therapeutic compound capable of treating depression in a patient is provided. The method comprises selecting a compound that significantly induces NR2B dependent synaptic plasticity.

### BRIEF DESCRIPTION OF THE FIGURES

FIG. 1 shows the effects of GLYX-13 and Ketamine on gene expression in the medial prefrontal cortex of an adult rat at 1 hour and 24 hours post-IV injection.

FIG. 2 shows the effects of GLYX-13 on Wnt pathway specific gene expression relative to vehicle controls.

FIG. 3 shows a schematic of the Wnt signaling pathway.

FIG. 4 shows the anti-depressant-like effects of GLYX-13 in multiple rat models.

FIG. 5 shows the results from various tests which indicate that GLYX-13 does not show ketamine-like addictive sensory-motor gating, or sedative side effects.

FIG. 6 shows the results of Porsolt tests which demonstrate that GLYX-13 is antidepressant-like compared to ketamine.

FIG. 7 shows results which demonstrate the GLYX-13 induces NR2B-dependent plasticity.

FIG. 8 shows the antidepressant-like effects of GLYX-13 are synaptic-plasticity related.

FIG. 9 shows GLYX-13 increases ex vivo [³H] MK-801 binding in rat medial prefrontal cortex 1 hour after dosing.

FIG. 10 shows plots of phosphoserine 1303 NR2B (pS1303 NR2B) protein levels (left panel) and total NR2B protein levels (middle panel) as a function of post-dosing time with GLYX-13 and shows a bar graph (right panel) of the ratio of pS1303 NR2B protein levels to total NR2B protein levels as a function of post-dosing time with GLYX-13.

FIG. 11 shows plots of phosphoserine 1480 NR2B (pS1480 NR2B) protein levels (left panel) and total NR2B protein levels (middle panel) as a function of post-dosing time with GLYX-13 and shows a bar graph (right panel) of the ratio of pS1480 NR2B protein levels to total NR2B protein levels as a function of post-dosing time with GLYX-13.

FIG. 12 shows a bar graph (left panel) of CK2 kinase activity 15 minutes post-dosing with GLYX-13 as measured by phosphorylation of a CK2 substrate and shows a plot (right panel) of a standard curve for the CK2 kinase activity assay.

### DETAILED DESCRIPTION

The present disclosure relates in part to methods of identifying a candidate compound suitable for treatment of depression. In some embodiments, a candidate compound may be a NMDAR partial agonist. In another aspect, the present disclosure relates in part to the use of identified compounds for treatment of clinically relevant depression and/or for general treatment of depression and/or anxiety.

Depression is a common psychological problem and refers to a mental state of low mood and aversion to activity. Various symptoms associated with depression include persistent anxious or sad feelings, feelings of helplessness, hopelessness, pessimism, and/or worthlessness, low energy, restlessness, irritability, fatigue, loss of interest in pleasurable activities or hobbies, excessive sleeping, overeating, appetite loss, insomnia, thoughts of suicide, and suicide attempts. The presence, severity, frequency, and duration of the above mentioned symptoms vary on a case to case basis. In some embodiments, a patient may have at least one, at least two, at least three, at least four, or at least five of these symptoms.

The most common depression conditions include Major Depressive Disorder and Dysthymic Disorder. Other depression conditions develop under unique circumstances. Such depression conditions include but are not limited to Psychotic depression, Postpartum depression, Seasonal affective disorder (SAD), mood disorder, depressions caused by chronic medical conditions such as cancer or chronic pain, chemotherapy, chronic stress, post traumatic stress disorders, and Bipolar disorder (or manic depressive disorder). Refractory depression occurs in patients suffering from depression who are resistant to standard pharmacological treatments, including tricyclic antidepressants, MAOIs, SSRIs, and double and triple uptake inhibitors and/or anxiolytic drugs, as well non-pharmacological treatments such as psychotherapy, electroconvulsive therapy, vagus nerve stimulation and/or transcranial magnetic stimulation. A treatment resistant-patient may be identified as one who fails to experience alleviation of one or more symptoms of depression (e.g., persistent anxious or sad feelings, feelings of helplessness, hopelessness, pessimism) despite undergoing one or more standard pharmacological or non-pharmacological treatment. In certain embodiments, a treatment-resistant patient is one who fails to experience alleviation of one or more symptoms of depression despite undergoing treatment with two different antidepressant drugs. In other embodiments, a treatment-resistant patient is one who fails to experience alleviation of one or more symptoms of depression despite undergoing treatment with four different antidepressant drugs. A treatment-resistant patient may also be identified as one who is unwilling or unable to tolerate the side effects of one or more standard pharmacological or non-pharmacological treatment. In certain embodiments, methods for treating refractory depression by administering an effective amount of an identified compound to a treatment-resistant patient in need thereof are contemplated. In an embodiment, methods of treating depression is contemplated when a patient has suffered depression for e.g. 5, 6, 7, 8 or more weeks, or for a month or more.

In an embodiment, a method for identifying a candidate compound suitable for treatment of depression is provided comprising exposing a cell to a potential compound in a culture medium, or administering a potential compound to an animal; retrieving a sample from the cell and/or culture medium, or from brain or neural tissue of the animal, at one or more predetermined time points; analyzing the sample for increased expression levels of Wnt1, and/or identifying the candidate compound as suitable for treatment of depression based on the increased expression level of Wnt1.

In another embodiment, a method for identifying a candidate compound suitable for treatment of depression, is provided comprising: exposing a cell to a potential compound in a culture medium, and/or or administering a potential compound to an animal; retrieving a sample from the cell and/or culture medium, or from brain or neural tissue of the animal, at one or more predetermined time points; analyzing the sample for increased expression levels of at least one of the genes listed in Table 1 or 2 (as provided below) indicated with a G, or decreased expression levels or at least one of the genes listed in Table 1 or 2 indicated with a K, and identifying the compound as suitable for treatment of depression based on the increased expression level or decreased expression level.

A contemplated sample may have a gene expression pattern as provided in Table 1 or 2 with the indication “G” and the identifying is based on increased expression of those genes.

Contemplated methods may further comprising analyzing the candidate compound for NDMA subunit NR2B synaptic plasticity.

A method for identifying a compound suitable for treatment of depression or other indications is provided herein in an embodiment is provided, wherein the method may include exposing a cell to a potential compound in a culture medium, or administering a potential compound to an animal; retrieving a sample from the cell and/or culture medium, or from brain or neural tissue of the animal, at one or more predetermined time points; analyzing the sample for NMDA receptor NR2B subunit plasticity, and identifying the compound as suitable for treatment of depression based on inducing the NR2B plasticity. A candidate compound suitable for treating depression may significantly induce NR2B dependent synaptic plasticity as compared to ketamine.

Tissues contemplated herein may be tissue of medial prefrontal cortex. Animals contemplated may be a rodent or human; cells may be a human or rodent cell. Contemplated candidate compounds may modulate a NMDA receptor, e.g. a candidate compound may be a NMDA partial agonist.

A candidate compound suitable for treating depression may have fewer side effects as compared to ketamine, for example the compound may not have substantial addictive sensory motor grating and/or sedative effect.

In an embodiment, a method of identifying a therapeutic compound capable of treating depression in a patient, is provided, comprising selecting a compound that significantly induces NR2B dependent synaptic plasticity.

Identified compounds may act predominantly at NR2B-containing NMDARs, and may not display the classic side effects of known NMDAR modulators such as CPC-101,606 and ketamine. For example, identified compounds may have markedly elevated long-term potentiation (LTP) while simultaneously reducing long-term depression (LTD) in rat hippocampal organotypic cultures. In some embodiments, identified compounds may produce an antidepressant effect essentially without dissociative side effects when administered to a subject in therapeutic amounts. In certain embodiments, an antidepressant effect with essentially no sedation may be produced by identified compounds when administered to a subject in therapeutic amounts. In still other embodiments, identified compounds may not have abuse potential (e.g., may not be habit-forming).

In some embodiments, compounds may increase AMPA GluR1 serine-845 phosphorylation or reduce expression in Wnt1 or Wnt signaling, for example as compared to ketamine.

Additionally, identified compounds may have better Blood-Brain Barrier (BBB) penetration as compared to many of the earlier glycine site ligands (Leeson & Iversen, J. Med. Chem. 37:4053-4067, 1994) and may cross the BBB readily. In some embodiments, identified compoudns or a composition comprising same may provide better i.v. in vivo potency and/or brain level concentration, relative to plasma levels, e.g. as compared to ketamine.

A variety of depression conditions are expected to be treated with an identified compound without for example affecting behavior or motor coordination, and without inducing or promoting seizure activity. Exemplary depression conditions that are expected to be treated according to this aspect include, but are not limited to, major depressive disorder, dysthymic disorder, psychotic depression, postpartum depression, premenstrual syndrome, premenstrual dysphoric disorder, seasonal affective disorder (SAD), anxiety, mood disorder, depressions caused by chronic medical conditions such as cancer or chronic pain, chemotherapy, chronic stress, post traumatic stress disorders, risk of suicide, and bipolar disorder (or manic depressive disorder). It should be understood that depression caused by bipolar disorder may be referred to as bipolar depression. In addition, patients suffering from any form of depression often experience anxiety. Various symptoms associated with anxiety include fear, panic, heart palpitations, shortness of breath, fatigue, nausea, and headaches among others. It is expected that the methods of the present condition can be used to treat anxiety or any of the symptoms thereof.

In addition, a variety of other neurological conditions are expected to be treated according to the methods. Exemplary conditions include, but are not limited to, a learning disorder, autistic disorder, attention-deficit hyperactivity disorder, Tourette's syndrome, phobia, post-traumatic stress disorder, dementia, AIDS dementia, Alzheimer's disease, Parkinson's disease, Huntington's disease, spasticity, myoclonus, muscle spasm, bipolar disorder, a substance abuse disorder, urinary incontinence, and schizophrenia.

Also provided herein are methods of treating depression in treatment resistant patients or treating refractory depression, e.g., patients suffering from a depression disorder that does not, and/or has not, responded to adequate courses of at least one, or at least two, other antidepressant compounds or therapeutics. For example, provided herein is a method of treating depression in a treatment resistant patient, comprising a) optionally identifying the patient as treatment resistant and b) administering an effective dose of an identified compound to said patient.

Symptoms of depression, and relief of same, may be ascertained by a physician or psychologist, e.g. by a mental state examination. Symptoms include thoughts of hopelessness, self-harm or suicide and/or an absence of positive thoughts or plans.

Contemplated methods include a method of treating autism and/or an autism spectrum disorder in a patient need thereof, comprising administering an effective amount of an identified to the patient. For example, upon administration, an identified compound may decrease the incidence of one or more symptoms of autism such as eye contact avoidance, failure to socialize, attention deficit, poor mood, hyperactivity, abnormal sound sensitivity, inappropriate speech, disrupted sleep, and perseveration. Such decreased incidence may be measured relative to the incidence in the untreated individual or an untreated individual(s). In some embodiments, patients suffering from autism also suffer from another medical condition, such as Fragile X syndrome, tuberous sclerosis, congenital rubella syndrome, and untreated phenylketonuria.

In another embodiment, methods of treating a disorder in a patient need thereof are contemplated, wherein the disorder is selected from group consisting of: epilepsy, AIDS dementia, multiple system atrophy, progressive supra-nuclear palsy, Friedrich's ataxia, autism, fragile X syndrome, tuberous sclerosis, attention deficit disorder, olivio-ponto-cerebellar atrophy, cerebral palsy, drug-induced optic neuritis, peripheral neuropathy, myelopathy, ischemic retinopathy, glaucoma, cardiac arrest, behavior disorders, and impulse control disorders that includes administering an identified compound.

In an embodiment, contemplated herein are methods of treating attention deficit disorder, ADHD (attention deficit hyperactivity disorder), schizophrenia, anxiety, amelioration of opiate, nicotine and/or ethanol addiction (e.g., method of treating such addiction or ameliorating the side effects of withdrawing from such addiction), spinal cord injury diabetic retinopathy, traumatic brain injury, post-traumatic stress syndrome and/or Huntington's chorea, in a patient in need thereof, that includes administering an identified compound. For example, patients suffering from schizophrenia, addiction (e.g. ethanol or opiate), autism, Huntington's chorea, traumatic brain injury, spinal cord injury, post-traumatic stress syndrome and diabetic retinopathy may all be suffering from altered NMDA receptor expression or functions.

In another embodiment, a method of treating Alzheimer's disease, or e.g., treatment of memory loss that e.g., accompanies early stage Alzheimer's disease, in a patient in need thereof is provided, comprising administering an identified compound.

Toxicity and therapeutic efficacy of subject compounds may be determined by standard pharmaceutical procedures in cell cultures or experimental animals, e.g., for determining the LD50 and the ED50.

As used herein, the term “GLYX peptide” refers to a peptide having NMDAR glycine-site partial agonist/antagonist activity. GLYX peptides may be obtained by well-known recombinant or synthetic methods such as those described in U.S. Pat. Nos. 5,763,393 and 4,086,196 herein incorporated by reference. In some embodiments, GLYX refers to a tetrapeptide having the amino acid sequence Thr-Pro-Pro-Thr (SEQ ID NO: 13), or L-threonyl-L-prolyl-L-prolyl-L-threonine amide. In some embodiments, candidate compounds have the same microarray results as GLYX-13 and/or the below compounds.

For example, GLYX-13 refers to the compound depicted as:

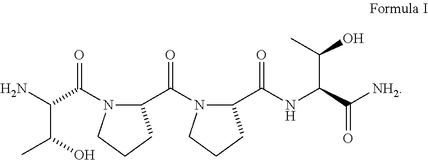

Also contemplated are polymorphs, homologs, hydrates, solvates, free bases, and/or suitable salt forms of GLYX 13 such as, but not limited to, the acetate salt. The peptide may be cyclyzed or non-cyclyzed form as further described in U.S. Pat. No. 5,763,393. In some embodiments, an a GLYX-13 analog may include an insertion or deletion of a moiety on one or more of the Thr or Pro groups such as a deletion of CH₂, OH, or NH₂ moiety. In other embodiments, GLYX-13 may be optionally substituted with one or more halogens, C₁-C₃ alkyl (optionally substituted with halogen or amino), hydroxyl, and/or amino Glycine-site partial agonist of the NMDAR are disclosed in U.S. Pat. No. 5,763,393, U.S. Pat. No. 6,107,271, and Wood et al., NeuroReport, 19, 1059-1061, 2008, the entire contents of which are herein incorporated by reference.

Candidate compounds may have substantially the same gene expression effect as one or more of the following compounds:

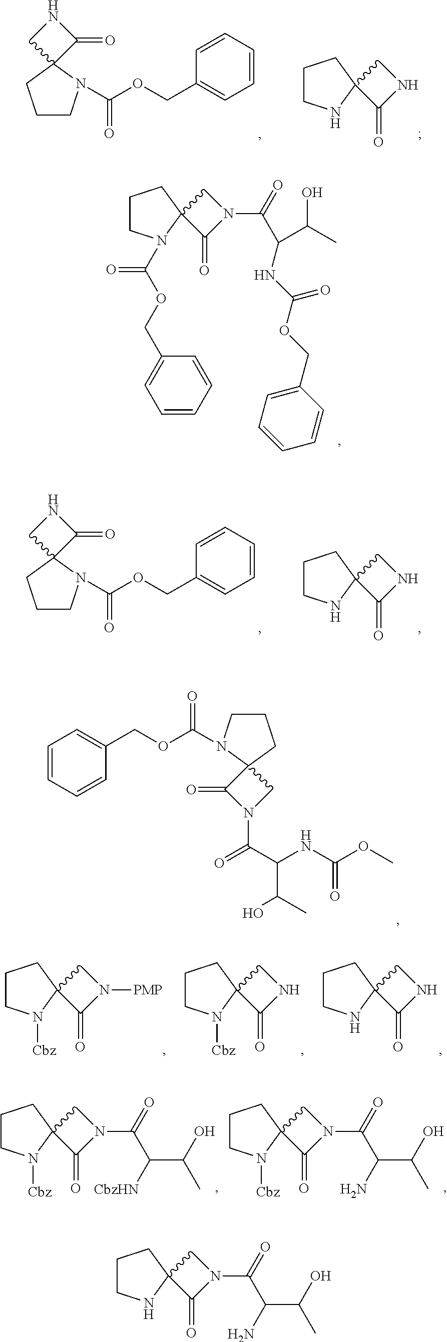

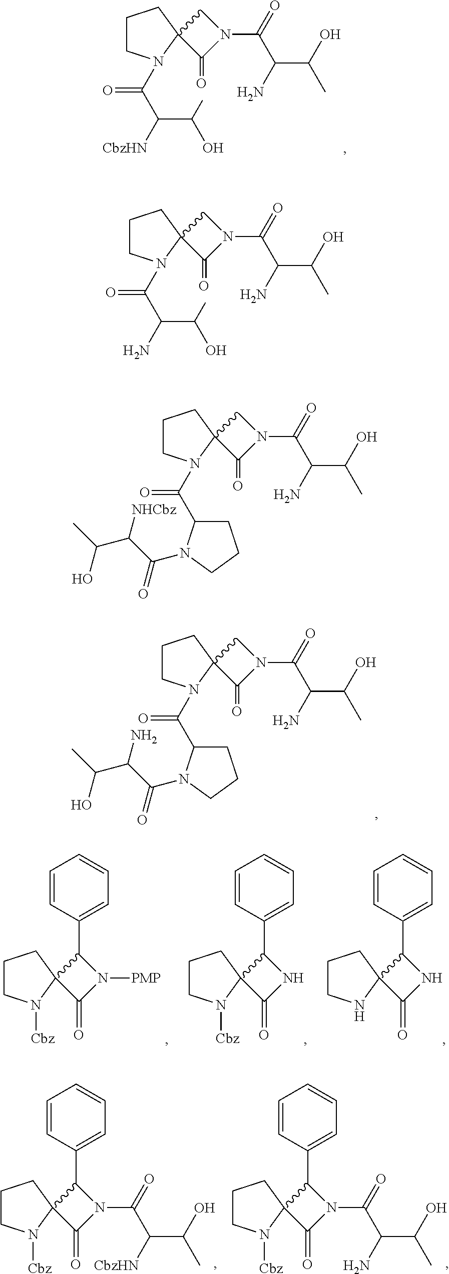

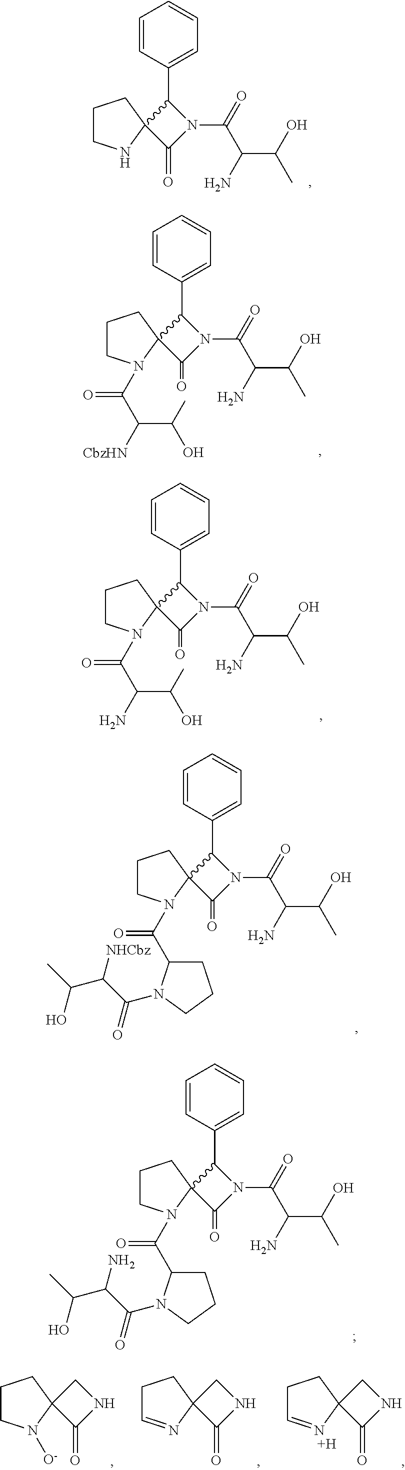

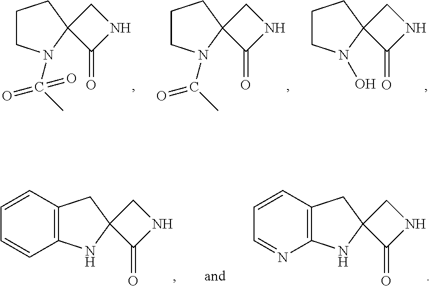

The present disclosure has multiple aspects, illustrated by the following non-limiting examples.

### EXAMPLES

### Example 1

### Gene Expression Patterns After Administration of GLYX-13 or Ketamine

In the present study, gene expression patterns in the medial prefrontal cortex (mPFC) were examined following either GLYX-13 (3 mg/kg, IV; the lowest dose that produces an anti-depressant effect in the Porsolt test) or ketamine (10 mg/kg, IV; a dose that produces a long-lasting anti-depressant effect in the Porsolt test) using a focused microarray platform combined with ontological analyses to identify functionally related gene sets that were differentially effected by GLYX-13 and ketamine Among the most interesting of these was the Wnt signaling pathway. A Wnt pathway-specific qRT-PCR array was used to corroborate these findings. Using this qRT-PCR array, the results showed that at 1 hr after GLYX-13 injections, 5 genes were differentially expressed as compared to saline treated control rats. At 24 hrs after GLYX-13 administration, 4 genes were upregulated. At 1 and 24 hrs following ketamine administration only 1 gene was downregulated. Taken together, these data suggest that although both GLYX-13 and ketamine produce rapid antidepressant-like effects in the Porsolt test, they likely effect changes in different cellular signaling pathways; one such example being the Wnt signaling pathway.

### Methods:

Animals:

Adult (2-3 month-old) male Sprague-Dawley rats (Harlan Laboratories, Indianapolis, Ind.) were housed 3 to a cage and injected (IV) with one of the following—GLYX-13 (3 mg/kg), ketamine (10 mg/kg), or saline vehicle (1 ml/kg). At 1 and 24 hours after injection (N=5 per time point for each treatment group), rats were sacrificed, their brains were quickly dissected, frozen, and then stored at −80° C. The medial prefrontal cortex (mPFC) was dissected from frozen tissue on ice. An equal volume of the homogenized tissue was used to extract and purify RNA for microarray analysis and to make cDNA for qRT-PCR analysis. All procedures were approved by the Northwestern University IACUC committee and performed in accordance with the NIH Guide for the Care and Use of Laboratory Animals.

Transcriptomics:

Using an in-house microarray (Kroes et al., 2006), we assayed the expression of 1,178 genes specific to the rat brain and representing more than >90% of the major gene ontological categories in the mPFC of rats at 1 or 24 hrs post-injection (IV) with GLYX-13, ketamine, or saline (N=5 adult male rats at each time-point per treatment group). Equivalent aliquots of rat reference RNA (Stratagene, La Jolla, Ca) were treated concurrently with the tissue samples. Reverse transcription of 5 ug of RNA (primed with an oligo(dT) primer bearing a T7 promoter was followed by in vitro transcription in the presence of amino-allyl dUTP. The aRNA was denatured, hybridized, and washed with high stringency. Fluorescence hybridization was then quantified by a high resolution confocal laser scanner utilizing QuantArray software and analyzed using GeneTraffic (Iobion Informatics, La Jolla, Calif.). Statistical analysis was performed using the permutation-based Significance Analysis of Microarrays (SAM) algorithm using a false discovery rate of <10%. We utilized Database for Annotation Visualization and Integrated Discovery (DAVID) gene functional classification and gene functional annotation tables to examine interrelated genes within the gene list obtained using SAM.

qRT-PCR Array:

Reverse transcription of 1.0 μg of DNAsed, total RNA from 4 rats was primed with oligo(dT) and random hexamers. We utilized SuperScriptIII according to manufacturer's specifications (Invitrogen, Carlsbad, Calif.). A 1:10 dilution of cDNA was used as a template for quantitative real-time PCR, and the analysis was performed with Brilliant SYBR Green qRT-PCR Master Mix (Stratagene) on a Mx3000P Real-Time PCR System. ROX reference dye was included in all reactions. Experiments were performed in triplicate for each data point and transcript abundance was normalized to reference genes included in the rat Wnt PCR array (Qiagen, 330231).

Results:

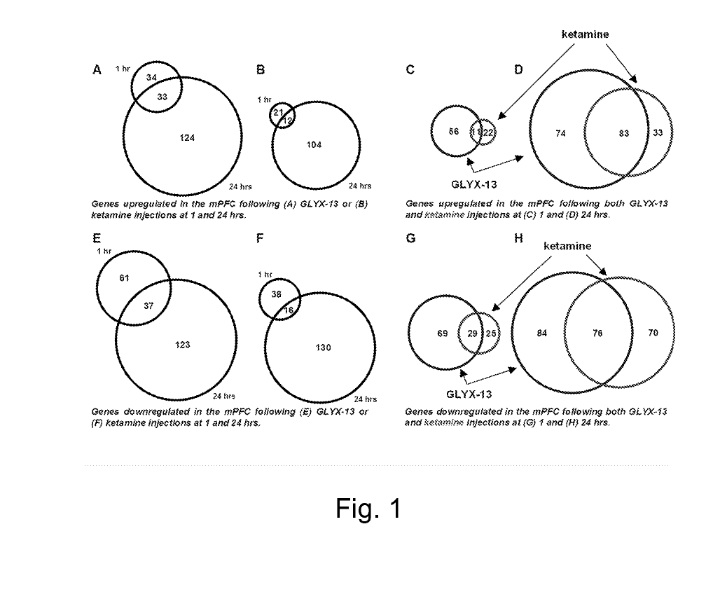

As shown in FIG. 1, GLYX-13 and ketamine differentially affect gene expression patterns in the mPFC of the adult rat at 1 and 24 hrs post-injection (IV). In FIG. 1, the numbers represent the total number of genes that were shown to be significantly differentially expressed at 1 and 24 hrs following either GLYX-13 or ketamine injections (IV) as compared to vehicle control rats, using SAM analysis (FDR <10%). The sample size for each group in FIG. 1 was 5 adult male rats.

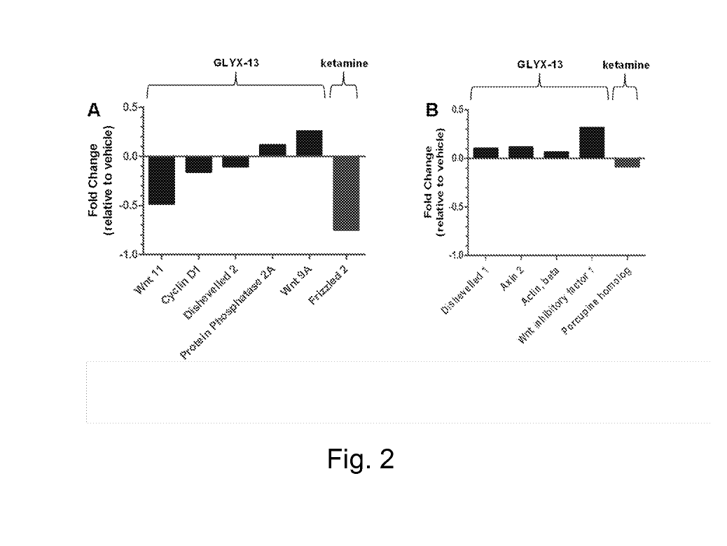

GLYX and ketamine showed a differential effect on the Wnt signaling pathway (Table 1 and FIG. 2). FIG. 2 shows Wnt signaling pathway gene expression in the mPFC following both GLYX-13 and ketamine injections at 1 hr (panel A) and 24 hrs (panel B). Using a commercially available rat Wnt qPCR array (Qiagen, 330231), a greater number of significant gene expression changes (p<0.05) were observed at 1 and 24 hrs post GLYX-13 injection relative to vehicle control rats when compared to the Wnt specific gene expression changes observed following ketamine injections. Fold change values greater than 0.0 indicate an upregulation in gene expression relative to saline vehicle controls, whereas those values less than 0.0 are genes whose expression was downregulated relative to vehicle controls. The sample size for each group in FIG. 2 was 4 rats. The significant reduction in the expression of Wnt11 in the mPFC at 1 hour post GLYX-13 injections suggest that the GLYX-13 mediated effect may, at least in part, involve a reduction in non-canonical Wnt signaling.

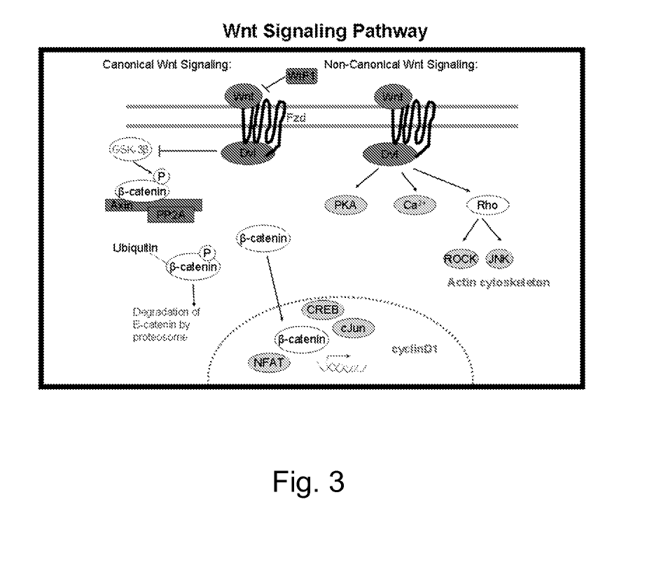

Notably, as shown in FIG. 3, GLYX-13 produced a greater number of changes in the Wnt pathway specific gene expression 1 and 24 hours post injection relative to vehicle control rats. Significant gene expression changes observed post-GLYX-13 injection are noted in dark gray, genes present on the qPCR array that did not significantly change are noted in light gray, and genes not present on the array are noted in medium gray. Significant gene expression changes following ketamine injections were observed for Frizzled 2 (Fzd).

Table 1. GLYX-13 and Ketamine differentially affect Wnt signaling pathway gene expression in the mPFC of the adult rat at 1 and 24 hours post injection (IV).

TABLE 1
Microarray Analysis of Wnt Signaling Pathway Gene Expression

| | | GLYX- | | | |
|---|---|---|---|---|---|
| | | 13 vs. | | Ketamine vs. | |
| | | Vehicle | | Vehicle | |
| Gene Bank Accession No. | Gene Name | 1 Hr | 24 Hr | 1 Hr | 24 Hr |
| NM_024405 | axin 1 | G | — | — | — |
| M16112 | calcium/calmodulin-dependent | — | V | — | — |
| | protein kinase II beta | | | | |
| NM_001042354 | calcium/calmodulin-dependent | — | G | — | K |
| | protein kinase II beta | | | | |
| NM_012519 | calcium/calmodulin-dependent | — | V | — | V |
| | protein kinase II delta | | | | |
| NM_053615 | casein kinase 1, alpha 1 | V | G | V | — |
| NM_053824 | casein kinase 2, alpha 1 | V | V | — | V |
| | polypeptide | | | | |
| NM_031021 | casein kinase 2, beta subunit | V | G | — | K |
| NM_053357 | catenin, beta 1 | V | G | V | V |
| NM_153474 | frizzled homolog 3 | — | — | — | V |
| X73653 | glycogen synthase kinase 3 beta | — | V | — | — |
| X07286 | PKC, alpha | — | V | V | — |
| K03486, X04139 | PKC, beta | — | G | — | K |
| L14323, M20636 | PLC, beta 1 | G | G | K | K |
| D90035, NM_017041 | Protein Phosphatase 2B | G | G | — | — |
| M31809 | Protein Phosphatase 3 | — | G | — | — |
| NM_001100922 | protein kinase, cAMP-dependent, | — | G | — | — |
| | catalytic, alpha | | | | |
| XM_224987 | secreted frizzled-related protein 1 | V | — | — | — |
| AB017912 | SMAD family member 2 | G | V | — | V |
| XM_235639 | Wnt 1 | G | V | — | — |
| XM_237296 | Wnt 10A | — | — | V | K |
| NM_053402 | Wnt 4 | — | — | V | — |
| XM_226051 | Wnt 8A | V | V | — | — |
| NM_053738 | Wnt inhibitory factor 1 | — | V | — | — |

Significance Analysis of Microarray Data (FDR < 10%).
G: indicative of higher levels of gene expression in GLYX-13 treated rats;
K: indicative of higher levels of gene expression in ketamine treated rats;
V: indicative of a higher level of expression in saline vehicle treated rats (downregulated in GLYX-13 or ketamine treated rats as indicated).
Genes present on the array that comprise the Wnt signaling pathway were defined using DAVID analysis.
N = 5 rats per group.

Data in Table 2 indicate the genes that were significantly differentially expressed in GLYX-13 and ketamine treated rats relative to either vehicle (GLYX-13 v Vehicle or Ketamine v Vehicle), or relative to each other (GLYX-13 v Ketamine) using significance analysis of microarrays (False Discovery Rate <10%). G: indicative of higher levels of gene expression in GLYX-13 treated rats; K: indicative of higher levels of gene expression in ketamine treated rats; V: indicative of a higher level of expression in saline vehicle treated rats (downregulated in GLYX-13 or ketamine treated rats as indicated). N=5 rats per group.

TABLE 2
Significance Analysis of Microarray Data for GLYX-13, Ketamine,
and vehicle control rats.

| | | | | | Ketamine v | | | Glyx v | | |
|---|---|---|---|---|---|---|---|---|---|---|
| | | Glyx v Vehicle | | | Vehicle | | | Ketamine | | |
| Gene Bank | | 1 | 24 | 2 | 1 | 24 | 2 | 1 | 24 | 2 |
| Accession No. | Gene Name | Hr | Hr | wks | Hr | Hr | wks | Hr | Hr | wks |
| A03913 | glia-derived neurite- | V | — | — | V | — | — | — | — | — |
| | promoting factor | | | | | | | | | |
| A17753 | D3 receptor | — | V | — | — | — | — | — | K | — |
| AB000280 | solute carrier family 15, | — | — | — | — | V | — | — | G | — |
| | member 4 | | | | | | | | | |
| AB002801 | cyclic nucleotide gated | — | V | — | — | — | — | — | K | — |
| | channel alpha 3 | | | | | | | | | |
| AB003478 | beta 1,3- | V | — | V | V | — | V | — | — | — |
| | galactosyltransferase, | | | | | | | | | |
| | polypeptide 4 | | | | | | | | | |
| AB003991, | SNAP 25 | G | G | — | — | — | K | G | G | K |
| AB003992 | | | | | | | | | | |
| AB006137 | galactoside 2-alpha-L- | — | — | G | — | — | — | — | — | — |
| | fucosyltransferase | | | | | | | | | |
| AB008682 | FGF 17 | — | — | — | — | V | — | — | — | — |
| AB010963, | calcium-activated potassium | — | G | — | — | K | — | — | — | — |
| AF020712 | channel beta subunit | | | | | | | | | |
| AB011679 | tubulin, beta 5 | — | V | — | K | K | — | K | — | — |
| AB013130 | myozenin 3; synaptopodin | — | V | — | — | V | — | K | G | — |
| AB015946 | tubulin, gamma 1 | — | V | — | V | — | V | — | — | — |
| AB016160 | GABA B receptor 1 | — | V | — | — | V | K | — | G | — |
| AB017656, | mAChR 3 | — | V | — | — | V | — | K | G | — |
| M16407, | | | | | | | | | | |
| M18088 | | | | | | | | | | |
| AB017912 | SMAD family member 2 | G | V | — | — | V | — | G | — | — |
| AB018049 | ST3 beta-galactoside alpha- | — | G | — | — | K | K | — | — | — |
| | 2,3-sialyltransferase 5 | | | | | | | | | |
| AB018546 | stress-associated | V | V | V | V | — | — | — | — | — |
| | endoplasmic reticulum | | | | | | | | | |
| | protein 1 | | | | | | | | | |
| AB020978 | growth arrest and DNA- | V | — | — | — | — | — | — | — | — |
| | damage-inducible, gamma | | | | | | | | | |
| AF000423 | synaptotagmin XI | — | V | — | — | — | — | — | — | — |
| AF000973 | potassium | — | V | V | — | — | V | — | — | — |
| | intermediate/small | | | | | | | | | |
| | conductance calcium- | | | | | | | | | |
| | activated channel, subfamily | | | | | | | | | |
| | N, member 1 | | | | | | | | | |
| AF001423 | NMDAR 2A | V | V | — | — | V | — | K | — | — |
| AF003598 | integrin, beta 7 | V | — | — | — | — | — | K | — | — |
| AF003825, | GDNF family receptor alpha | V | — | — | — | V | — | — | G | — |
| U97143 | 2 | | | | | | | | | |
| AF005720 | chloride channel 2 | — | V | — | — | V | — | — | — | — |
| AF007583 | acetylcholinesterase | — | V | — | — | — | — | — | — | — |
| AF007758, | synuclein, alpha | — | G | G | — | — | K | — | G | — |
| S73007 | | | | | | | | | | |
| AF012347 | SMAD family member 9 | — | G | V | — | — | — | — | — | — |
| AF013144 | dual specificity phosphatase | V | V | V | V | V | — | — | — | — |
| | 5 | | | | | | | | | |
| AF015728 | cyclic nucleotide gated | — | — | — | — | — | — | K | — | — |
| | channel beta 1 | | | | | | | | | |
| AF017637 | carboxypeptidase Z | V | — | — | — | — | — | K | — | — |
| AF019973 | enolase 2, gamma, neuronal | — | G | G | — | K | — | — | — | — |
| AF021935 | CDC42 binding protein | — | — | — | — | V | — | — | G | G |
| | kinase alpha | | | | | | | | | |
| AF022819 | potassium channel, | — | — | — | — | V | — | — | G | — |
| | subfamily K, member 1 | | | | | | | | | |
| AF022935 | prolactin | — | V | — | — | — | — | — | — | — |
| AF025670 | caspase 6 | — | — | G | — | — | K | G | — | — |
| AF025671 | caspase 2 | — | G | — | — | K | — | — | G | — |
| U77933 | | | | | | | | | | |
| AF027984 | calcium channel, voltage- | — | G | — | — | K | — | — | — | G |
| | dependent, T type, alpha 1G | | | | | | | | | |
| | subunit | | | | | | | | | |
| AF028784 | glial fibrillary acidic protein | — | — | — | — | — | — | G | — | — |
| AF030086 | neurotransmitter-induced | — | V | G | — | V | K | — | K | — |
| | early gene 1 (ania-1) | | | | | | | | | |
| AF030087 | activity and | — | — | — | — | K | — | — | K | — |
| | neurotransmitter-induced | | | | | | | | | |
| | early gene 2 (ania-2) mRNA | | | | | | | | | |
| AF030088 | homer homolog 1 | — | — | — | K | — | — | — | — | — |
| AF030089 | doublecortin-like kinase 1 | — | — | — | — | K | — | — | — | — |
| AF030091 | cyclin L1 | V | V | — | — | — | — | — | K | — |
| AF030253 | GABA vesicular transporter | — | — | G | — | — | K | — | — | — |
| AF030358 | chemokine (C-X3-C motif) | — | G | — | — | — | K | — | — | — |
| | ligand 1 | | | | | | | | | |
| AF031384 | potassium channel, | — | — | G | — | — | — | — | — | G |
| | subfamily K, member 3 | | | | | | | | | |
| AF031522 | galanin receptor 3 | — | — | G | — | — | — | — | — | — |
| AF032872 | protein inhibitor of activated | — | — | — | — | V | — | — | G | — |
| | STAT, 3 | | | | | | | | | |
| AF035632 | syntaxin 12 | — | — | — | — | V | — | — | G | — |
| AF037067 | TNF superfamily, member 4 | — | V | — | — | V | — | — | — | — |
| AF037071 | NOS 1, neuronal | G | — | — | K | V | K | G | G | K |
| X59949 | | | | | | | | | | |
| AF038571 | solute carrier family 1 | — | V | — | — | — | — | — | K | — |
| | (neuronal/epithelial high | | | | | | | | | |
| | affinity glutamate | | | | | | | | | |
| | transporter, system Xag), | | | | | | | | | |
| | member 1 | | | | | | | | | |
| AF041244 | hypocretin (orexin) receptor | — | — | — | — | K | — | — | — | — |
| | 1 | | | | | | | | | |
| AF041373 | phosphatidylinositol binding | — | — | — | — | — | — | K | — | — |
| | clathrin assembly protein | | | | | | | | | |
| AF042499 | SMAD family member 7 | — | — | — | — | V | — | — | — | — |
| AF042713 | neurexophilin 3 | G | — | — | — | V | — | — | — | — |
| AF042714 | neurexophilin 4 | V | — | — | — | — | — | — | — | — |
| AF044581 | syntaxin 12 | — | G | — | — | K | — | — | — | — |
| AF044910 | survival motor neuron 1 | — | V | — | — | V | — | — | — | — |
| AF048828 | voltage-dependent anion | V | — | — | V | — | — | — | — | — |
| | channel 1 | | | | | | | | | |
| AF049239 | sodium channel, voltage | — | V | G | — | V | — | — | — | G |
| | gated, type VIII, alpha | | | | | | | | | |
| | subunit | | | | | | | | | |
| AF049344 | UDP-N-acetyl-alpha-D- | — | V | — | — | V | — | K | — | — |
| | galactosamine:polypeptide | | | | | | | | | |
| | N- | | | | | | | | | |
| | acetylgalactosaminyl- | | | | | | | | | |
| | transferase 5 (GalNAc-T5) | | | | | | | | | |
| AF049882 | Cd82 molecule | — | V | — | V | V | — | — | — | — |
| AF050659 | activity and | — | V | — | — | — | — | K | — | — |
| | neurotransmitter-induced | | | | | | | | | |
| | early gene 7 (ania-7) mRNA | | | | | | | | | |
| AF050660 | activity and | — | V | — | — | — | — | — | K | — |
| | neurotransmitter-induced | | | | | | | | | |
| | early gene 8 (ania-8) mRNA | | | | | | | | | |
| AF050661 | activity and | — | — | — | — | V | — | — | — | — |
| | neurotransmitter-induced | | | | | | | | | |
| | early gene 9 (ania-9) mRNA | | | | | | | | | |
| AF050663 | activity and | G | — | G | — | — | — | — | — | G |
| | neurotransmitter-induced | | | | | | | | | |
| | early gene 11 (ania-11) | | | | | | | | | |
| AF052596 | SNAP 23 | — | V | — | — | — | — | — | — | — |
| AF054586 | ring finger protein 112V | V | — | — | — | — | K | K | — | |
| AF057308 | hypoxia-inducible factor 1, | — | G | V | — | K | — | — | — | — |
| | alpha subunit | | | | | | | | | |
| AF058795 | GABA B receptor 2 | — | G | — | — | K | K | — | — | K |
| AF060879 | neurocan | — | V | — | — | — | — | — | — | — |
| AF064541, | arginine vasopressin | — | V | — | — | V | — | — | — | — |
| D45400, | receptor 1B | | | | | | | | | |
| U27322 | | | | | | | | | | |
| AF076167 | UDP-N-acetyl-alpha-D- | — | — | — | — | — | V | — | — | G |
| | galactosamine:polypeptide | | | | | | | | | |
| | N- | | | | | | | | | |
| | acetylgalactosaminyltransfer- | | | | | | | | | |
| | ase 7 (GalNAc-T7) | | | | | | | | | |
| AF078779 | sodium leak channel, non- | G | G | — | K | K | — | — | G | — |
| | selective | | | | | | | | | |
| AF081366 | potassium inwardly-rectifying | — | — | — | — | — | — | — | G | — |
| | channel, subfamily J, | | | | | | | | | |
| | member 1 | | | | | | | | | |
| AF081557 | glial cells missing homolog 1 | — | — | G | — | — | — | — | — | G |
| | (Drosophila) | | | | | | | | | |
| AF087431 | glucosidase 1 | — | — | — | — | K | — | — | — | — |
| AF087453 | potassium voltage-gated | — | V | — | — | V | — | — | — | — |
| | channel, subfamily Q, | | | | | | | | | |
| | member 2 | | | | | | | | | |
| AF087454 | potassium voltage-gated | — | V | — | — | — | — | — | K | — |
| | channel, subfamily Q, | | | | | | | | | |
| | member 3 | | | | | | | | | |
| AF087839 | ATP-binding cassette, sub- | — | V | — | — | — | — | — | — | — |
| | family C (CFTR/MRP), | | | | | | | | | |
| | member 9 | | | | | | | | | |
| AF089730 | potassium channel, | — | G | — | — | — | — | — | — | — |
| | subfamily T, member 1 | | | | | | | | | |
| AF090113 | glutamate receptor | — | — | — | — | V | — | — | — | — |
| | interacting protein 2 | | | | | | | | | |
| AF096291 | Bcl2-like 2; poly(A) binding | — | — | — | — | — | — | G | — | — |
| | protein, nuclear 1 | | | | | | | | | |
| AF159803 | Non-specific lipid-transfer | — | — | — | — | K | — | — | — | — |
| | protein 6 | | | | | | | | | |
| AF191028 | Xylem cysteine proteinase 2 | — | — | — | — | V | — | — | G | — |
| AF205717 | transmembrane 4 L six | — | V | — | — | — | — | — | — | — |
| | family member 4 | | | | | | | | | |
| AF247559 | Triosephosphate isomerase, | V | V | — | — | — | — | — | — | — |
| | chloroplastic; | | | | | | | | | |
| | Triosephosphate isomerase | | | | | | | | | |
| AF264018 | transferase | — | V | — | — | — | — | — | — | — |
| AF459021 | tubulin, beta 3 | G | G | G | V | K | K | G | — | — |
| AJ000515 | cyclic nucleotide gated | G | V | — | — | — | — | G | — | — |
| | channel beta 1 | | | | | | | | | |
| AJ000556 | Janus kinase 1 | V | — | — | — | V | — | K | — | — |
| AJ001029 | SRY (sex determining region | V | — | — | V | — | — | — | — | — |
| | Y)-box 10 | | | | | | | | | |
| AJ002942 | retinoic acid receptor, beta | — | — | — | — | V | — | — | G | — |
| AJ003065 | potassium inwardly-rectifying | — | — | — | — | V | — | — | — | — |
| | channel, subfamily J, | | | | | | | | | |
| | member 14 | | | | | | | | | |
| AJ006519 | amiloride-sensitive cation | — | — | — | — | V | — | — | — | — |
| | channel 2, neuronal | | | | | | | | | |
| AJ006710 | phosphoinositide-3-kinase, | — | — | — | — | V | — | — | G | — |
| | class 3 | | | | | | | | | |
| AJ006855 | synaptojanin 1 | G | — | G | — | — | K | — | — | — |
| AJ007627, | potassium voltage-gated | — | V | V | — | — | — | — | K | K |
| AJ007628 | channel, subfamily H (eag- | | | | | | | | | |
| | related), member 4 | | | | | | | | | |
| AJ007632 | potassium voltage-gated | — | V | — | — | V | — | — | — | — |
| | channel, subfamily H (eag- | | | | | | | | | |
| | related), member 8 | | | | | | | | | |
| AJ012603 | ADAM metallopeptidase | — | — | V | — | — | V | — | — | — |
| | domain 17 | | | | | | | | | |
| AJ222813, | interleukin 18 | — | — | — | — | K | — | — | K | — |
| U77776 | | | | | | | | | | |
| AJ277828 | endothelial PAS domain | — | V | — | — | V | — | — | — | — |
| | protein 1 | | | | | | | | | |
| AJ295749 | xylosyltransferase II | — | — | — | — | V | — | — | — | — |
| D00634 | adrenergic, beta-1-, receptor | — | G | — | — | — | — | — | — | G |
| D00698, | IGF 1 | V | V | — | V | — | V | K | K | G |
| M15480, | | | | | | | | | | |
| M15481, | | | | | | | | | | |
| X06107 | | | | | | | | | | |
| D00833, | glycine receptor, alpha 1 | — | V | — | — | V | — | — | — | — |
| NM_013133 | | | | | | | | | | |
| D10106 | platelet-derived growth | — | G | V | — | K | V | — | — | — |
| | factor alpha polypeptide | | | | | | | | | |
| D12519 | syntaxin 1A | — | G | — | — | — | K | — | G | K |
| D12524 | v-kit Hardy-Zuckerman 4 | — | V | — | — | V | — | — | — | — |
| | feline sarcoma viral | | | | | | | | | |
| | oncogene homolog | | | | | | | | | |
| D12573 | hippocalcin | G | — | — | — | K | — | — | — | — |
| D13212, | NMDAR 2C | — | G | — | — | K | K | G | — | — |
| NM_012575, | | | | | | | | | | |
| U08259 | | | | | | | | | | |
| D13417 | hairy and enhancer of split 1 | — | — | — | — | — | V | — | K | G |
| D13418 | hairy and enhancer of split 3 | G | — | — | — | — | — | G | — | — |
| D13871 | solute carrier family 2 | — | V | — | — | V | — | — | — | — |
| | (facilitated glucose/fructose | | | | | | | | | |
| | transporter), member 5 | | | | | | | | | |
| D13985 | chloride channel, nucleotide- | V | — | — | V | — | — | — | — | — |
| | sensitive, 1A | | | | | | | | | |
| D14048 | heterogeneous nuclear | — | G | V | V | K | — | G | — | — |
| | ribonucleoprotein U | | | | | | | | | |
| D14480 | calpain 8 | — | — | — | — | V | — | K | — | — |
| D14869, | prostaglandin E receptor 3 | V | V | — | V | V | — | — | — | — |
| D16443 | (subtype EP3) | | | | | | | | | |
| D17469 | thyrotropin releasing | — | — | — | — | V | — | — | — | — |
| | hormone receptor | | | | | | | | | |
| D17521 | chloride channel 3 | — | V | — | — | V | — | G | — | — |
| D17764 | synuclein, beta | — | G | G | — | — | K | — | G | — |
| D25224 | similar to 40S ribosomal | V | V | G | — | — | K | K | — | — |
| | protein SA | | | | | | | | | |
| D25233 | retinoblastoma 1 | — | G | G | — | K | — | G | — | — |
| D25290 | cadherin 6 | V | V | — | — | V | — | — | — | — |
| D26111 | chloride channel Kb | — | V | — | — | — | — | — | — | — |
| D26154 | RB109 | — | — | — | V | — | — | G | — | — |
| D28560 | ectonucleotide | — | — | G | — | — | K | — | — | — |
| | pyrophosphatase/phospho- | | | | | | | | | |
| | diesterase 2 | | | | | | | | | |
| D28562 | solute carrier family 2 | — | V | — | — | V | — | — | — | — |
| | (facilitated glucose/fructose | | | | | | | | | |
| | transporter), member 5 | | | | | | | | | |
| D30666 | acyl-CoA synthetase long- | — | G | — | — | — | K | — | — | — |
| | chain family member 3 | | | | | | | | | |
| D32045, | adrenergic, alpha-1B-, | G | — | — | — | V | — | — | — | — |
| M60655 | receptor | | | | | | | | | |
| D32249 | praja 2, RING-H2 motif | — | G | G | — | K | — | G | — | G |
| | containing | | | | | | | | | |
| D38380 | signal recognition particle | — | G | V | V | — | — | G | G | — |
| | receptor, B subunit; | | | | | | | | | |
| | transferrin | | | | | | | | | |
| D44495 | APEX nuclease | — | — | V | V | V | V | G | G | — |
| | (multifunctional DNA repair | | | | | | | | | |
| | enzyme) 1 | | | | | | | | | |
| D45187 | cathepsin E | — | V | — | — | — | — | — | — | — |
| D50093 | prion protein | — | G | — | — | — | — | — | G | — |
| D50497 | chloride channel 5 | — | — | — | — | V | — | — | G | — |
| D63772 | solute carrier family 1 | — | — | V | — | — | — | — | — | K |
| | (neuronal/epithelial high | | | | | | | | | |
| | affinity glutamate | | | | | | | | | |
| | transporter, system Xag), | | | | | | | | | |
| | member 1 | | | | | | | | | |
| D84450 | ATPase, Na⁺/K⁺ | — | — | G | — | — | K | — | — | — |
| | transporting, beta 3 | | | | | | | | | |
| | polypeptide | | | | | | | | | |
| D86039 | potassium inwardly rectifying | — | V | — | — | V | — | — | — | — |
| | channel, subfamily J, | | | | | | | | | |
| | member 11 | | | | | | | | | |
| D87839 | 4-aminobutyrate | — | V | — | — | V | — | G | — | — |
| | aminotransferase | | | | | | | | | |
| D88672 | phospholipase D2 | — | — | — | K | — | — | — | — | — |
| D89655 | scavenger receptor class B, | — | — | G | — | — | — | — | — | G |
| | member 1 | | | | | | | | | |
| | protein phosphatase 3 | | | | | | | | | |
| D90035, | (formerly 2B), catalytic | G | G | G | — | — | K | G | G | G |
| NM_017041 | subunit, alpha isoform | | | | | | | | | |
| D90048, | ATPase, Na⁺/K⁺ | V | V | G | — | V | — | K | G | — |
| J04629 | transporting, beta 2 | | | | | | | | | |
| | polypeptide | | | | | | | | | |
| D90258 | proteasome subunit, alpha | — | G | — | — | — | — | — | — | — |
| | type 3 | | | | | | | | | |
| E00988 | IGF II | — | V | — | — | V | — | — | — | — |
| E01789 | rat C-kinase type-II (beta-2) | — | G | — | — | — | — | — | G | — |
| E01884 | rat IL-1-beta(interleukin-1 | — | — | — | — | — | — | — | G | — |
| | beta). | | | | | | | | | |
| E05646 | rat hippocampal cholinergic | V | — | — | V | V | — | — | — | — |
| | neurostimulating | | | | | | | | | |
| E12625 | novel protein which is | — | — | — | V | — | V | — | — | — |
| | expressed with nerve injury | | | | | | | | | |
| E12746 | Rat cholecystokinin-A | — | V | — | — | — | — | — | — | — |
| | receptor gene | | | | | | | | | |
| E13644 | Neurodap-1 | G | G | — | — | — | — | G | — | G |
| E13732 | CC chemokine receptor | — | V | — | — | V | — | — | — | — |
| | protein | | | | | | | | | |
| J02722 | heme oxygenase (decycling) | V | — | — | — | — | — | — | — | — |
| | 1 | | | | | | | | | |
| J03624 | galanin prepropeptide | — | G | — | K | — | — | K | — | — |
| J03754 | ATPase, Ca⁺⁺ transporting, | V | — | — | V | — | — | — | — | K |
| | plasma membrane 2 | | | | | | | | | |
| J03933 | thyroid hormone receptor | — | — | — | V | — | — | — | — | — |
| | beta | | | | | | | | | |
| J04024 | ATPase, Ca⁺⁺ transporting, | — | G | — | — | K | — | — | — | — |
| | cardiac muscle, slow twitch | | | | | | | | | |
| | 2 | | | | | | | | | |
| J04532 | PKC, zeta | — | G | — | — | — | — | — | G | — |
| J04563, | phosphodiesterase 4B, | — | V | — | — | V | — | — | — | — |
| M25350 | cAMP specific | | | | | | | | | |
| J04625 | carboxypeptidase E | — | G | — | — | K | — | G | — | — |
| J04636 | nACh receptor, beta 3 | — | G | — | — | — | — | — | G | — |
| J04731 | potassium voltage-gated | G | — | G | — | — | — | G | — | G |
| | channel, shaker-related | | | | | | | | | |
| | subfamily, member 2 | | | | | | | | | |
| J04811 | growth hormone receptor | — | V | — | — | V | — | — | — | — |
| J04963 | carcinoembryonic antigen- | V | V | — | — | — | — | — | — | — |
| | related cell adhesion | | | | | | | | | |
| | molecule 1 (biliary | | | | | | | | | |
| | glycoprotein) | | | | | | | | | |
| J05107 | hydroxysteroid 11-beta | V | — | V | — | — | — | — | — | K |
| | dehydrogenase 1 | | | | | | | | | |
| J05122 | translocator protein | V | V | — | — | — | — | K | K | — |
| J05510 | IP3 receptor, type 1 | — | G | G | — | — | — | — | — | G |
| K00512 | myelin basic protein | G | — | — | — | — | — | G | — | — |
| K01701 | oxytocin, prepropeptide | G | — | — | K | — | — | — | — | — |
| K03486 | PKC, beta | — | G | G | — | K | K | — | — | — |
| X04139 | | | | | | | | | | |
| L02926 | interleukin 10 | — | V | — | — | V | — | — | — | — |
| L04535 | somatostatin receptor 5 | — | — | — | V | — | V | — | — | — |
| L04684 | calcium channel, voltage- | — | — | — | — | V | — | — | — | — |
| | dependent, L type, alpha 1S | | | | | | | | | |
| | subunit | | | | | | | | | |
| L04739 | ATPase, Ca++ transporting, | G | G | — | — | — | K | G | — | K |
| | plasma membrane 1 | | | | | | | | | |
| L04796 | glucagon receptor | — | V | — | — | V | — | G | K | — |
| L05435 | synaptic vesicle glycoprotein | — | G | — | — | — | K | — | G | — |
| | 2a | | | | | | | | | |
| L05596 | serotonin receptor 1F | G | — | — | — | — | — | G | — | — |
| L06894 | platelet-derived growth | — | V | — | — | V | — | — | — | — |
| | factor alpha polypeptide | | | | | | | | | |
| L08228, | NMDAR 1 | G | G | V | K | — | — | G | — | — |
| X63255 | | | | | | | | | | |
| L08492 | GABA A receptor, alpha 3 | V | — | — | V | — | — | — | G | — |
| L08493 | GABA A receptor, alpha 4 | — | — | — | — | — | — | — | G | — |
| L08497 | GABA A receptor, gamma 2 | — | G | — | — | — | K | — | G | — |
| L09119 | neurogranin | — | G | — | — | — | K | — | — | K |
| L09120 | calpain 2 | — | V | — | — | — | — | — | — | — |
| L10072 | serotonin receptor 5A | — | V | — | — | V | K | — | — | — |
| L10326 | GNAS complex locus | — | — | — | — | — | K | G | — | — |
| L14323, | PLC, beta 1 | G | G | G | K | K | — | — | — | — |
| M20636 | | | | | | | | | | |
| L14851 | neurexin 3 | V | — | — | V | V | — | — | G | — |
| L16532 | 2′,3′-cyclic nucleotide 3′ | — | — | — | V | — | — | G | — | — |
| | phosphodiesterase | | | | | | | | | |
| L16764 | heat shock 70 kD protein 1B | — | — | — | K | — | — | K | — | — |
| L18889 | calnexin | — | — | — | — | V | — | — | — | — |
| L20821 | syntaxin 4 | — | V | — | — | — | — | — | — | — |
| L20822 | syntaxin 5 | V | V | — | V | V | — | — | G | — |
| L21192, | GAP 43 | G | G | G | — | K | K | G | G | K |
| NM_017195 | | | | | | | | | | |
| L22339 | sulfotransferase family, | — | — | — | — | — | V | — | — | — |
| | cytosolic, 1C, member 3 | | | | | | | | | |
| L23088 | selectin, platelet | — | — | — | V | — | — | — | — | — |
| L24907, | calcium/calmodulin | — | G | — | — | V | K | — | G | K |
| NM_134468 | dependent protein kinase I | | | | | | | | | |
| L25925 | prostaglandin-endoperoxide | — | V | — | — | — | — | — | K | — |
| | synthase 2 | | | | | | | | | |
| L27059 | phosphodiesterase 4D, | — | — | — | — | V | — | — | — | — |
| | cAMP-specific | | | | | | | | | |
| L27487 | calcitonin receptor-like V | V | — | — | V | — | K | — | — | |
| L31546 | serotonin receptor 2A | G | G | — | — | K | K | — | — | — |
| L31620 | nAChR, alpha 4 | — | — | — | K | — | — | — | — | — |
| L31622 | nAChR, beta 2 | — | — | — | — | K | — | — | — | — |
| L31771 | adrenergic, alpha-1D-, | — | V | — | — | — | — | — | G | — |
| M60654 | receptor | | | | | | | | | |
| L36884 | protein tyrosine | — | V | — | — | V | — | — | — | — |
| | phosphatase, receptor type, | | | | | | | | | |
| | V | | | | | | | | | |
| L41623 | platelet-derived growth | — | V | — | — | — | — | — | — | — |
| | factor beta polypeptide | | | | | | | | | |
| M10088 | prodynorphin | — | — | — | K | V | — | K | — | — |
| M10244 | tyrosine hydroxylase | — | — | — | — | K | — | — | — | — |
| M11794 | metallothionein 2A | V | V | — | V | — | — | — | — | — |
| M12492 | protein kinase, cAMP | — | G | — | — | K | — | — | — | K |
| | dependent regulatory, type II | | | | | | | | | |
| | beta | | | | | | | | | |
| M15191 | tachykinin 1 | — | G | — | — | — | — | — | — | — |
| M15880 | neuropeptide Y | — | G | — | — | K | — | — | — | — |
| M16112 | calcium/calmodulin- | — | V | — | — | — | — | — | — | — |
| | dependent protein kinase II | | | | | | | | | |
| | beta | | | | | | | | | |
| M16406 | mAChR 1 | — | G | — | — | — | — | G | — | — |
| M16409 | mAChR 4 | — | — | — | — | — | K | — | — | — |
| M17069, | calmodulin | — | G | — | — | K | V | — | G | — |
| X13817, | | | | | | | | | | |
| X14265 | | | | | | | | | | |
| M17526 | G protein alpha activating | G | — | — | — | — | K | G | — | K |
| | activity polypeptide O | | | | | | | | | |
| M18416 | early growth response 1 | G | — | — | K | K | — | — | K | — |
| M19257 | retinol binding protein 1, | G | — | — | — | — | — | — | — | — |
| | cellular | | | | | | | | | |
| M19533 | peptidylprolyl isomerase A | V | G | — | — | K | — | — | — | — |
| M20133 | androgen receptor | G | — | G | — | — | — | K | — | G |
| M20636 | PLC, beta 1 | V | — | — | — | — | — | — | — | — |
| M21060, | SOD 1 | V | G | V | — | K | V | — | — | — |
| M25157, | | | | | | | | | | |
| Y00404 | | | | | | | | | | |
| M22253 | sodium channel, voltage- | — | G | — | K | — | — | — | G | — |
| | gated, type I, alpha | | | | | | | | | |
| M22254 | sodium channel, voltage- | — | G | G | — | — | K | — | G | — |
| | gated, type II, alpha 1 | | | | | | | | | |
| M22357 | myelin-associated | G | G | — | — | — | K | — | — | — |
| | glycoprotein | | | | | | | | | |
| M22412 | potassium voltage-gated | — | V | — | — | — | — | — | K | — |
| | channel, Isk-related | | | | | | | | | |
| | subfamily, member 1 | | | | | | | | | |
| M23601 | monoamine oxidase B | — | — | V | — | — | — | — | — | K |
| M23697 | plasminogen activator, | — | — | — | — | — | — | — | — | K |
| | tissue | | | | | | | | | |
| M24104 | vesicle-associated | — | V | — | — | — | K | — | — | — |
| | membrane protein 1 | | | | | | | | | |
| M24852 | Purkinje cell protein 4 | — | G | — | — | K | — | G | — | — |
| M25638 | neurofilament, light | — | G | — | — | — | — | — | G | — |
| | polypeptide | | | | | | | | | |
| M25646 | arginine vasopressin | — | — | — | — | V | V | — | — | — |
| M25888 | proteolipid protein 1 | G | G | — | — | — | K | G | G | K |
| M25890 | somatostatin | — | G | — | — | K | — | — | — | K |
| M26161 | potassium voltage-gated | G | G | — | — | — | — | G | — | — |
| | channel, shaker-related | | | | | | | | | |
| | subfamily, member 1 | | | | | | | | | |
| M26715 | phosphodiesterase 4A, | — | G | — | — | — | — | — | G | — |
| | cAMP-specific | | | | | | | | | |
| M26744, | interleukin 6 | — | V | — | V | V | — | — | — | — |
| M26745 | | | | | | | | | | |
| M27293 | IGF 1 receptor | — | — | — | — | V | — | — | — | — |
| M27925 | synapsin II | — | G | — | — | — | — | — | G | — |
| M30312 | potassium voltage-gated | — | V | G | — | V | K | — | — | — |
| | channel, shaker-related | | | | | | | | | |
| | subfamily, member 3 | | | | | | | | | |
| M31174, | thyroid hormone receptor | — | G | — | — | K | — | G | — | — |
| X12744 | alpha | | | | | | | | | |
| M31176 | gastrin releasing peptide | — | — | — | — | V | — | — | G | — |
| M31178 | calbindin 1 | — | G | — | K | K | — | — | — | — |
| M31433 | | — | — | — | — | V | — | — | G | — |
| M31809 | protein phosphatase 3, | — | G | G | — | — | K | — | G | — |
| | catalytic subunit, beta | | | | | | | | | |
| | isoform | | | | | | | | | |
| M32061 | adrenergic, alpha-2B-, | — | V | — | — | V | — | — | — | — |
| | receptor | | | | | | | | | |
| M34253 | interferon regulatory factor 1 | — | V | — | — | V | — | — | — | — |
| M34445 | glutamate decarboxylase 1 | — | G | — | K | K | K | — | — | — |
| M35162, | GABA A receptor, delta | — | — | G | — | — | — | K | — | G |
| NM_017289 | | | | | | | | | | |
| M36074 | nuclear receptor subfamily 3, | — | — | — | — | V | — | — | G | — |
| | group C, member 2 | | | | | | | | | |
| M36831, | D2 receptor | V | — | V | V | — | — | G | — | — |
| X56065, | | | | | | | | | | |
| NM_012547 | | | | | | | | | | |
| M37394 | epidermal growth factor | — | V | — | — | V | — | — | — | — |
| | receptor | | | | | | | | | |
| M38061 | AMPA 2 receptor | G | G | G | — | K | K | G | G | — |
| M55291 | neurotrophic tyrosine kinase, | G | — | G | — | V | K | G | G | — |
| | receptor, type 2 | | | | | | | | | |
| M57664 | creatine kinase, brain | — | G | — | — | K | — | — | — | — |
| M58040 | transferrin receptor | — | G | — | — | K | — | G | — | — |
| M58316 | adrenergic, alpha-2C-, | G | — | — | — | — | — | — | G | — |
| X57659 | receptor | | | | | | | | | |
| M58634 | IGF binding protein 1 | — | V | — | — | — | — | K | — | — |
| M59313, | potassium voltage gated | — | G | V | — | V | K | — | — | K |
| X62839 | channel, Shaw-related | | | | | | | | | |
| | subfamily, member 2 | | | | | | | | | |
| M59980 | potassium voltage gated | — | V | — | — | — | — | — | — | — |
| | channel, Shal-related family, | | | | | | | | | |
| | member 2 | | | | | | | | | |
| M60525, | VGF nerve growth factor | G | G | — | K | K | — | — | — | K |
| M74223 | inducible | | | | | | | | | |
| M60753 | catechol-O- | V | — | — | V | — | — | — | — | — |
| | methyltransferase | | | | | | | | | |
| M61099 | mGluR 1 | — | G | — | — | — | — | — | — | — |
| M61177, | MAPK 3 | V | — | — | — | K | — | — | — | — |
| NM_017347 | | | | | | | | | | |
| M62781 | IGF binding protein 5 | — | V | — | — | V | — | — | — | — |
| M63101 | IL 1 receptor antagonist | — | — | — | — | V | — | — | G | — |
| M63122 | tumor necrosis factor | — | V | — | — | — | — | — | K | — |
| | receptor superfamily, | | | | | | | | | |
| | member 1a | | | | | | | | | |
| M64300 | MAPK 1 | — | — | — | — | V | — | — | G | — |
| M68971 | hexokinase 2 | — | — | — | — | — | K | — | K | — |
| M69055 | IGF binding protein 6 | V | V | — | — | — | — | — | K | — |
| M77482 | potassium voltage gated | — | V | — | — | — | — | — | — | — |
| | channel, Shab-related | | | | | | | | | |
| | subfamily, member 2 | | | | | | | | | |
| M77809 | transforming growth factor, | — | — | — | — | V | — | G | G | — |
| | beta receptor III | | | | | | | | | |
| M80545 | calcium channel, voltage- | V | — | — | — | — | — | — | — | — |
| | dependent, beta 2 subunit | | | | | | | | | |
| M81783 | potassium voltage-gated | — | — | — | — | V | — | — | G | — |
| | channel, subfamily F, | | | | | | | | | |
| | member 1 | | | | | | | | | |
| M82824 | NF1 | — | — | — | K | V | — | — | — | — |
| M84009, | D4 receptor | G | V | G | K | V | — | — | — | G |
| NM_012944 | | | | | | | | | | |
| M84725 | transgelin 3 | — | G | — | — | — | K | — | G | — |
| M85035 | AMPA 2 | V | V | — | V | V | — | — | — | — |
| M85183 | NLR family, pyrin domain | — | — | — | — | — | — | G | — | — |
| | containing 6 | | | | | | | | | |
| M86389, | heat shock protein 1 | V | V | V | — | K | — | K | K | — |
| S45392, | | | | | | | | | | |
| X54793 | | | | | | | | | | |
| M86621 | calcium channel, voltage- | G | G | — | — | — | K | G | G | K |
| | dependent, alpha2/delta | | | | | | | | | |
| | subunit 1 | | | | | | | | | |
| M86742 | neurotrophin 4 | — | V | — | — | — | — | — | — | — |
| M86835 | vasoactive intestinal peptide | — | — | — | V | — | K | G | — | K |
| | receptor 1 | | | | | | | | | |
| M88096 | cholecystokinin A receptor | — | V | — | — | — | — | — | — | — |
| M88751 | calcium channel, voltage- | — | — | — | — | V | — | — | — | — |
| | dependent, beta 3 subunit | | | | | | | | | |
| M89924 | calcium channel, voltage- | — | — | G | — | — | K | — | — | — |
| | dependent, L type, alpha 1C | | | | | | | | | |
| | subunit | | | | | | | | | |
| M89953 | serotonin receptor 1D | — | V | — | — | V | — | — | — | — |
| M89954, | serotonin receptor 1B | — | — | — | — | K | K | — | — | — |
| X62944 | | | | | | | | | | |
| M91466 | adenosine A2B receptor | — | V | — | — | — | — | — | — | — |
| M91599 | FGFR 4 | — | V | — | — | — | — | — | — | — |
| M91652 | glutamate-ammonia ligase | — | G | — | — | K | K | — | — | K |
| | (glutamine synthetase) | | | | | | | | | |
| M91808 | sodium channel, voltage- | — | — | — | — | V | — | — | G | — |
| | gated, type I, beta | | | | | | | | | |
| M92075 | mGluR 2 | G | G | — | — | K | — | — | — | — |
| M92076 | mGluR 3 | — | — | — | — | V | — | — | — | — |
| M92905 | calcium channel, voltage- | G | G | — | — | — | — | G | — | — |
| | dependent, N type, alpha 1B | | | | | | | | | |
| | subunit | | | | | | | | | |
| M95735 | syntaxin 1B | — | — | — | K | — | — | K | — | — |
| M95762 | solute carrier family 6 | — | — | — | K | — | — | — | — | — |
| | (neurotransmitter | | | | | | | | | |
| | transporter, GABA), member | | | | | | | | | |
| | 13 | | | | | | | | | |
| M96375 | neurexin 1 | — | — | — | K | K | K | — | — | — |
| M96376 | neurexin 2 | — | G | — | — | K | K | — | — | — |
| M96853 | discs, large homolog 4 | — | — | — | — | — | K | — | — | — |
| | (Drosophila) | | | | | | | | | |
| M98820 | interleukin 1 beta | — | V | — | — | V | — | — | — | — |
| NM_001003 | DNA (cytosine-5-)- | V | — | V | V | — | V | — | — | — |
| 964 | methyltransferase 3-like | | | | | | | | | |
| NM_001007 | catenin (cadherin associated | V | — | V | — | K | — | K | K | — |
| 145 | protein), alpha 1 | | | | | | | | | |
| NM_001008 | eukaryotic translation | — | G | G | — | K | K | — | — | — |
| 335 | initiation factor 4A2 | | | | | | | | | |
| NM_001017 | eukaryotic translation | — | G | — | — | K | — | — | G | — |
| 374 | initiation factor 4, gamma 2, | | | | | | | | | |
| | pseudogene 1 | | | | | | | | | |
| NM_001024 | catenin, beta like 1 | — | — | — | — | K | — | — | K | — |
| 870 | | | | | | | | | | |
| NM_001024 | exocyst complex component | — | G | V | — | — | — | — | — | K |
| 964 | 3 | | | | | | | | | |
| NM_001033 | eukaryotic translation | — | V | — | — | V | V | — | — | — |
| 069 | initiation factor 4E binding | | | | | | | | | |
| | protein 2 | | | | | | | | | |
| NM_001033 | casein kinase 1, gamma 2 | — | — | V | — | — | — | — | — | — |
| 870 | | | | | | | | | | |
| NM_001042 | calcium/calmodulin- | — | G | — | — | K | — | — | — | — |
| 354 | dependent protein kinase II | | | | | | | | | |
| | beta | | | | | | | | | |
| NM_001100 | eukaryotic translation | — | — | — | — | — | V | — | G | G |
| 158 | initiation factor 4A, isoform 3 | | | | | | | | | |
| NM_001100 | protein kinase, cAMP- | — | G | — | — | — | — | — | — | — |
| 922 | dependent, catalytic, alpha | | | | | | | | | |
| NM_001104 | Eph receptor B1 | G | G | — | — | — | K | — | G | K |
| 528 | | | | | | | | | | |
| NM_001106 | AKT1 substrate 1 (proline- | G | — | — | — | — | — | G | — | — |
| 259 | rich) | | | | | | | | | |
| NM_001106 | catenin (cadherin associated | — | G | — | — | K | K | — | — | — |
| 598 | protein), alpha 2 | | | | | | | | | |
| NM_001106 | eukaryotic translation | — | G | — | — | K | K | — | — | — |
| 693 | initiation factor 4 gamma, 3 | | | | | | | | | |
| NM_001106 | chromobox homolog 5 (HP1 | G | — | — | — | — | — | — | — | — |
| 797 | alpha homolog, Drosophila) | | | | | | | | | |
| NM_001107 | adaptor-related protein | — | G | — | — | K | K | — | — | — |
| 511 | complex 2, alpha 1 subunit | | | | | | | | | |
| NM_001108 | SHC (Src homology 2 | — | G | — | — | K | — | — | — | — |
| 065 | domain containing) | | | | | | | | | |
| | transforming protein 2 | | | | | | | | | |
| NM_001108 | protein phosphatase 2A | — | G | V | — | K | — | — | — | K |
| 577 | activator, regulatory subunit | | | | | | | | | |
| | 4 | | | | | | | | | |
| NM_001108 | eukaryotic translation | — | — | — | — | — | — | — | K | — |
| 808 | initiation factor 4E member 2 | | | | | | | | | |
| NM_001109 | c-fos serum response | — | G | — | — | — | — | — | — | — |
| 302 | element-binding | | | | | | | | | |
| | transcription factor | | | | | | | | | |
| NM_001130 | Transforming protein p21 | — | G | — | — | K | — | — | G | — |
| 441 | | | | | | | | | | |
| NM_001134 | regulatory associated | — | — | — | — | K | K | — | — | K |
| 499 | protein of MTOR, complex 1 | | | | | | | | | |
| NM_001135 | son of sevenless homolog 2 | — | G | V | — | K | V | — | — | — |
| 561 | | | | | | | | | | |
| NM_012512 | beta-2 microglobulin | V | — | V | — | K | V | K | — | — |
| NM_012519 | calcium/calmodulin- | — | V | — | — | V | — | — | — | — |
| | dependent protein kinase II | | | | | | | | | |
| | delta | | | | | | | | | |
| NM_012528 | nAChR, beta 1 (muscle) | — | V | — | — | — | — | — | — | — |
| NM_012734 | hexokinase 1 | — | G | — | — | K | K | G | — | — |
| NM_012752 | CD24 molecule | — | — | — | — | K | — | — | — | K |
| NM_012813 | ST8 alpha-N-acetyl- | — | — | — | — | V | — | — | G | — |
| | neuraminide alpha-2,8- | | | | | | | | | |
| | sialyltransferase 1 | | | | | | | | | |
| NM_012821 | adenylate cyclase 6 | — | — | — | — | — | K | — | — | — |
| NM_012957, | GABA A receptor, beta 2 | G | G | — | — | — | — | G | — | — |
| X15467 | | | | | | | | | | |
| NM_013058 | inhibitor of DNA binding 3 | V | — | — | — | — | — | K | — | — |
| NM_013060 | inhibitor of DNA binding 2 | — | G | — | — | K | K | — | — | — |
| NM_013180 | integrin beta 4 | — | V | — | — | V | — | — | — | — |
| NM_013216 | Ras homolog enriched in | G | G | V | — | K | K | — | — | K |
| | brain | | | | | | | | | |
| NM_017022 | integrin beta 1 (fibronectin | V | — | — | — | K | — | K | — | — |
| | receptor beta) | | | | | | | | | |
| NM_017035 | PLC, delta 1 | G | — | — | — | — | — | G | — | — |
| NM_017066 | pleiotrophin | — | G | — | K | K | K | K | — | K |
| NM_017078 | nAChR, alpha 5 | — | — | — | K | V | — | — | — | — |
| NM_017093 | v-akt murine thymoma viral | — | — | — | — | V | — | — | — | — |
| | oncogene homolog 2 | | | | | | | | | |
| NM_017125 | Cd63 molecule | V | — | V | — | — | — | K | K | — |
| NM_017291 | GABA receptor, rho 1 | — | — | — | — | V | — | — | — | — |
| NM_019218 | neurogenic differentiation 1 | — | G | — | — | — | K | — | G | — |
| NM_019220 | amino-terminal enhancer of | — | G | — | — | K | — | — | — | — |
| | split | | | | | | | | | |
| NM_019248 | neurotrophic tyrosine kinase, | — | — | — | — | — | — | — | — | K |
| | receptor, type 3 | | | | | | | | | |
| NM_019361 | activity-regulated | — | V | — | — | V | K | — | — | K |
| | cytoskeleton-associated | | | | | | | | | |
| | protein | | | | | | | | | |
| NM_021576 | 5′ nucleotidase, ecto | — | V | — | — | V | — | — | — | G |
| NM_021835, | Jun oncogene | — | — | — | — | — | V | — | — | — |
| X17163 | | | | | | | | | | |
| NM_021836 | jun B proto-oncogene | V | V | — | — | — | — | K | K | — |
| NM_022282 | discs, large homolog 2 | — | — | — | — | — | — | — | K | — |
| | (Drosophila) | | | | | | | | | |
| NM_022855 | casein kinase 1, gamma 3 | — | G | — | — | K | K | — | — | — |
| NM_022952 | adaptor-related protein | — | — | V | — | — | — | — | — | — |
| | complex 2, sigma 1 subunit | | | | | | | | | |
| NM_024405 | axin 1 | G | — | V | — | — | — | — | — | — |
| NM_031007 | adenylate cyclase 2 | G | — | — | K | — | — | — | G | — |
| NM_031008 | adaptor-related protein | G | — | V | — | — | — | G | — | — |
| | complex 2, alpha 2 subunit | | | | | | | | | |
| NM_031021 | casein kinase 2, beta | V | G | — | — | K | — | — | — | — |
| | subunit | | | | | | | | | |
| NM_031338 | calcium/calmodulin- | — | — | — | — | — | K | — | — | — |
| | dependent protein kinase | | | | | | | | | |
| | kinase 2, beta | | | | | | | | | |
| NM_031515, | v-Ki-ras2 Kirsten rat | V | V | — | — | V | — | — | — | — |
| U09793 | sarcoma viral oncogene | | | | | | | | | |
| | homolog | | | | | | | | | |
| NM_031527 | protein phosphatase 1, | — | G | V | — | K | — | — | — | — |
| | catalytic subunit, alpha | | | | | | | | | |
| | isoform | | | | | | | | | |
| NM_031575 | v-akt murine thymoma viral | V | V | — | — | V | — | K | — | — |
| | oncogene homolog 3 | | | | | | | | | |
| | (protein kinase B, gamma) | | | | | | | | | |
| NM_031590 | WNT1 inducible signaling | — | V | — | — | — | — | — | K | — |
| | pathway protein 2 | | | | | | | | | |
| NM_031622 | MAPK 6 | V | G | — | — | — | — | — | — | — |
| NM_031639 | discs, large homolog 3 | — | — | V | — | K | — | — | K | K |
| | (Drosophila) | | | | | | | | | |
| NM_031662 | calcium/calmodulin- | — | G | — | — | — | K | — | — | — |
| | dependent protein kinase | | | | | | | | | |
| | kinase 1, alpha | | | | | | | | | |
| NM_031985 | ribosomal protein S6 kinase, | — | — | — | — | K | — | — | — | — |
| | 70 kDa, polypeptide 1 | | | | | | | | | |
| NM_032058 | eukaryotic translation | V | G | V | — | — | V | — | G | — |
| | initiation factor 2B, subunit 2 | | | | | | | | | |
| | beta | | | | | | | | | |
| NM_033230 | v-akt murine thymoma viral | — | — | V | — | K | — | — | K | — |
| | oncogene homolog 1 | | | | | | | | | |
| NM_053346, | neuritin 1 | G | G | — | — | K | K | G | — | K |
| U88958 | | | | | | | | | | |
| NM_053351 | calcium channel, voltage- | G | G | — | K | — | K | — | G | K |
| | dependent, gamma subunit | | | | | | | | | |
| | 2 | | | | | | | | | |
| NM_053354 | DNA (cytosine-5-)- | G | — | — | — | K | K | — | K | — |
| | methyltransferase 1 | | | | | | | | | |
| NM_053357 | catenin (cadherin associated | V | G | V | V | V | — | K | G | — |
| | protein), beta 1 | | | | | | | | | |
| NM_053400 | transducin-like enhancer of | V | — | — | — | — | — | K | — | — |
| | split 3 (E(sp1) homolog, | | | | | | | | | |
| | Drosophila) | | | | | | | | | |
| NM_053402 | wingless-type MMTV | — | — | — | V | — | — | G | — | — |
| | integration site family, | | | | | | | | | |
| | member 4 | | | | | | | | | |
| NM_053615 | casein kinase 1, alpha 1 | V | G | — | V | — | — | — | G | — |
| NM_053649 | kringle containing | — | — | V | — | V | V | — | G | — |
| | transmembrane protein 1 | | | | | | | | | |
| NM_053698 | Cbp/p300-interacting | — | — | — | K | K | V | K | K | — |
| | transactivator, with Glu/Asp- | | | | | | | | | |
| | rich carboxy-terminal | | | | | | | | | |
| | domain, 2 | | | | | | | | | |
| NM_053738 | Wnt inhibitory factor 1 | — | V | — | — | — | — | — | — | — |
| NM_053824 | casein kinase 2, alpha 1 | V | V | — | — | V | — | — | — | — |
| | polypeptide | | | | | | | | | |
| NM_053837 | adaptor-related protein | — | G | V | — | — | — | — | — | — |
| | complex 2, mu 1 subunit | | | | | | | | | |
| NM_053857 | eukaryotic translation | V | — | V | — | — | V | — | K | G |
| | initiation factor 4E binding | | | | | | | | | |
| | protein 1 | | | | | | | | | |
| NM_053861 | tenascin C | — | — | — | — | — | V | — | — | — |
| NM_080394 | reelin | — | G | — | — | K | — | — | — | — |
| NM_080583 | adaptor-related protein | — | G | — | — | K | — | — | — | — |
| | complex 2, beta 1 subunit | | | | | | | | | |
| NM_130779 | adenylate cyclase 3 | G | — | — | — | K | — | — | — | — |
| NM_133605 | calcium/calmodulin- | — | — | — | — | — | K | — | — | K |
| | dependent protein kinase II | | | | | | | | | |
| | gamma | | | | | | | | | |
| NM_133609 | eukaryotic translation | G | — | — | — | — | — | — | — | — |
| | initiation factor 2B, subunit 3 | | | | | | | | | |
| | gamma | | | | | | | | | |
| NM_138905 | phosphatidic acid | G | G | — | — | K | — | G | — | — |
| | phosphatase type 2B | | | | | | | | | |
| NM_139060 | casein kinase 1, delta | — | G | G | — | K | K | — | — | — |
| NM_153474 | frizzled homolog 3 | — | — | V | — | V | — | — | — | K |
| NM_172029 | eukaryotic translation | — | V | — | — | V | — | G | G | — |
| | initiation factor 2B, subunit 1 | | | | | | | | | |
| | alpha | | | | | | | | | |
| NM_173331 | MAPK 15 | — | V | — | — | — | — | — | — | — |
| NM_173337 | calcium/calmodulin- | — | G | G | — | — | K | G | G | — |
| | dependent protein kinase II | | | | | | | | | |
| | inhibitor 1 | | | | | | | | | |
| NM_182842 | calcium/calmodulin- | — | G | — | — | — | — | — | G | — |
| | dependent protein kinase IG | | | | | | | | | |
| NM_199372 | eukaryotic translation | — | — | — | — | K | — | — | — | — |
| | initiation factor 4A1 | | | | | | | | | |
| S42358 | neurotransmitter transporter, | — | G | — | — | — | — | — | — | — |
| | GABA, member 11 | | | | | | | | | |
| S44606 | beta-integrin | V | — | V | — | — | — | K | — | — |
| S48813 | adrenergic, beta, receptor | — | G | — | — | K | — | — | — | — |
| | kinase 1 | | | | | | | | | |
| S49491 | proenkephalin 1 | G | G | — | — | — | V | — | — | — |
| S53527 | S100 calcium binding | G | G | — | — | K | — | G | G | — |
| | protein B | | | | | | | | | |
| S54008 | FGFR 1 | — | — | — | — | — | K | — | — | — |
| S59158, | glial high affinity glutamate | G | G | — | — | K | K | G | — | — |
| X63744 | transporter, member 3 | | | | | | | | | |
| S62043 | serotonin receptor 6 | — | V | — | — | — | — | — | — | — |
| S67770 | transforming growth factor, | — | V | — | — | V | — | — | — | — |
| | beta receptor II | | | | | | | | | |
| S68944 | solute carrier family 6 | — | — | — | — | — | — | — | — | G |
| | (neurotransmitter | | | | | | | | | |
| | transporter), member 17 | | | | | | | | | |
| S70690, | cholecystokinin | — | G | — | — | K | K | — | — | K |
| X01032 | | | | | | | | | | |
| S71570 | calcium/calmodulin- | — | — | — | — | — | K | — | — | K |
| | dependent protein kinase II | | | | | | | | | |
| | gamma | | | | | | | | | |
| S72505, | glutathione S-transferase A3 | — | — | — | — | V | — | G | — | — |
| X78848 | | | | | | | | | | |
| S75952 | glucagon-like peptide 1 | — | — | — | — | V | — | — | — | — |
| | receptor | | | | | | | | | |
| S76145 | neurotransmitter transporter, | — | — | G | — | — | K | — | — | — |
| | dopamine, member 3 | | | | | | | | | |
| S76779 | apolipoprotein E | G | G | — | — | — | K | G | — | — |
| S77528, | CCAAT/enhancer binding | V | V | V | K | — | — | K | K | — |
| X60769 | protein (C/EBP), beta | | | | | | | | | |
| S79263 | colony stimulating factor 2 | — | — | — | — | V | — | K | G | — |
| | receptor, beta, low-affinity | | | | | | | | | |
| | (granulocyte-macrophage) | | | | | | | | | |
| S82649, | neuronal pentraxin 2 | — | V | — | — | V | K | — | — | — |
| XM_221901 | | | | | | | | | | |
| S83194 | calcium/calmodulin- | — | G | — | — | — | K | — | G | K |
| | dependent protein kinase | | | | | | | | | |
| | kinase 1, alpha | | | | | | | | | |
| U03390 | G protein, beta polypeptide | V | V | V | — | — | V | K | — | — |
| | 2 like 1 | | | | | | | | | |
| U04738 | somatostatin receptor 4 | — | — | — | — | V | — | — | G | — |
| U05784 | microtubule-associated | — | G | — | — | K | — | — | — | — |
| | protein 1 light chain 3 beta | | | | | | | | | |
| U08141 | Ferritin light chain 2 | — | — | — | — | V | — | — | G | — |
| U08255 | glutamate receptor, | V | G | V | — | — | — | — | — | — |
| | ionotropic, delta 1 | | | | | | | | | |
| U08256 | glutamate receptor, | V | V | — | — | — | — | — | K | — |
| | ionotropic, delta 2 | | | | | | | | | |
| U08260 | NMDAR 2D | — | G | — | — | — | — | — | — | — |
| U08290 | neuronatin | — | G | — | — | K | K | — | — | — |
| U10071 | CART prepropeptide | — | — | — | V | — | — | — | — | — |
| U11031 | contactin 3 | — | V | — | — | V | — | — | K | — |
| U12336 | nAChR, alpha 9 | — | — | — | — | — | K | — | — | K |
| U13368 | adrenergic, alpha-1A-, | — | V | — | — | — | K | — | — | — |
| | receptor | | | | | | | | | |
| U14533 | nuclear receptor subfamily 1, | V | — | — | — | K | — | — | — | — |
| | group H, member 2 | | | | | | | | | |
| U15211 | retinoic acid receptor, alpha | — | V | — | — | — | — | — | — | — |
| U17254 | nuclear receptor subfamily 4, | — | — | — | K | K | — | K | K | — |
| | group A, member 1 | | | | | | | | | |
| U17607 | nuclear transcription factor-Y | — | — | — | — | — | V | — | — | — |
| | gamma | | | | | | | | | |
| U18650 | huntingtin | — | V | — | — | V | — | — | — | G |
| U18982 | FBJ osteosarcoma | — | — | — | — | — | K | — | — | — |
| | oncogene B | | | | | | | | | |
| U20105 | synaptotagmin VI | — | V | — | — | — | — | — | K | — |
| U20283 | syntaxin binding protein 2 | V | — | — | — | — | — | — | — | — |
| U21662 | mannosyl (alpha-l,6-)- | V | G | — | — | K | — | — | — | — |
| | glycoprotein beta-1,2-N- | | | | | | | | | |
| | acetylglucosaminyl- | | | | | | | | | |
| | transferase | | | | | | | | | |
| U22414 | chemokine (C—C motif) | V | — | — | — | — | — | — | — | — |
| | ligand 3 | | | | | | | | | |
| U26402 | synaptotagmin V | — | V | — | — | V | — | — | — | — |
| U26541 | PDGFA associated protein 1 | — | G | — | V | — | — | — | — | — |
| U29701 | 4-aminobutyrate | — | G | — | — | — | K | — | G | — |
| | aminotransferase | | | | | | | | | |
| U30290 | galanin receptor 1 | V | V | — | — | V | — | K | — | — |
| U30938, | microtubule-associated | — | G | — | — | K | K | — | — | K |
| X53455 | protein 2 | | | | | | | | | |
| U31203 | noggin | — | — | — | — | — | — | — | G | — |
| U31554 | limbic system-associated | — | G | — | — | V | — | — | G | — |
| | membrane protein | | | | | | | | | |
| U33472 | serine/threonine kinase 10 | — | V | — | V | — | — | — | K | — |
| U35365 | FYN oncogene related to | — | — | — | — | V | — | — | — | — |
| | SRC, FGR, YES | | | | | | | | | |
| U37058 | neuromedin B receptor | — | — | — | — | V | — | — | — | — |
| U37142 | brevican | V | V | — | — | — | — | K | — | — |
| U38306 | arylalkylamine N- | — | V | — | — | — | — | — | K | — |
| | acetyltransferase | | | | | | | | | |
| U38653 | IP3 receptor, type 1 | — | V | — | — | V | — | — | — | — |
| U48829, | NOS 2 | — | V | V | — | V | — | K | — | — |
| S71597, | | | | | | | | | | |
| U03699, | | | | | | | | | | |
| U16359 | | | | | | | | | | |
| U49729 | Bcl2-associated X protein | V | — | — | — | — | — | — | — | — |
| U49953 | p21 protein (Cdc42/Rac)- | — | — | G | — | — | K | — | — | — |
| | activated kinase 1 | | | | | | | | | |
| U50147 | discs, large homolog 3 | — | — | — | — | V | — | — | G | — |
| | (Drosophila) | | | | | | | | | |
| U50194 | tripeptidyl peptidase II | — | G | — | K | — | — | — | — | — |
| U52948 | complement component 9 | — | — | — | V | — | — | — | — | — |
| U53927 | solute carrier family 7 | V | — | — | — | — | K | K | — | — |
| | (cationic amino acid | | | | | | | | | |
| | transporter, y⁺ system), | | | | | | | | | |
| | member 2 | | | | | | | | | |
| U57715 | FGF receptor activating | V | V | — | V | V | — | — | — | — |
| | protein 1 | | | | | | | | | |
| U59809 | IGF 2 receptor | G | — | V | — | — | V | G | — | — |
| U61696 | arginyl aminopeptidase | — | — | — | V | V | — | G | G | — |
| | (aminopeptidase B) | | | | | | | | | |
| U63740 | fasciculation and elongation | V | G | — | — | — | K | — | G | — |
| | protein zeta 1 (zygin I) | | | | | | | | | |
| U67140 | discs, large homolog- | — | G | V | — | — | — | — | G | — |
| | associated protein 4 | | | | | | | | | |
| U72350 | similar to Bcl2-like 1 isoform | — | — | — | V | — | — | — | — | — |
| | 3; Bcl2-like 1 | | | | | | | | | |
| U72353 | lamin B1 | — | V | — | — | — | — | — | — | — |
| U73142 | MAPK 14 | G | V | — | — | — | — | G | — | — |
| U73859 | hexokinase 3 | — | V | — | — | — | — | G | — | |
| U75899 | heat shock protein 2 | — | V | — | — | V | — | — | — | — |
| U81492 | interleukin 3 | V | V | — | V | V | — | — | — | — |
| U83112 | forkhead box M1 | V | G | V | — | — | — | — | G | — |
| U88036 | solute carrier organic anion | — | G | — | — | — | — | — | G | — |
| | transporter family, member | | | | | | | | | |
| | 1a4 | | | | | | | | | |
| U88324, | G protein, beta polypeptide | V | G | V | V | K | K | — | — | K |
| AF022083 | 1 | | | | | | | | | |
| U92469 | gonadotropin releasing | V | — | — | — | — | — | K | — | — |
| | hormone receptor | | | | | | | | | |
| U92655 | potassium voltage-gated | G | G | — | K | K | — | — | — | — |
| | channel, subfamily Q, | | | | | | | | | |
| | member 1 | | | | | | | | | |
| V01217 | actin, beta | V | G | — | — | K | — | K | — | — |
| V01227 | tubulin, alpha | V | G | V | V | K | — | G | G | — |
| X00336 | interferon alpha family | — | G | — | — | — | K | — | — | K |
| X00911, | IGF 2 | G | G | — | — | K | K | G | — | K |
| X16703, | | | | | | | | | | |
| X17012 | | | | | | | | | | |
| X01454 | thyroid stimulating hormone, | — | — | — | — | V | K | — | — | — |
| | beta | | | | | | | | | |
| X02231 | GAPDH | V | V | G | V | V | — | K | G | G |
| X03347 | FBR-murine osteosarcoma | V | — | — | — | — | — | — | — | — |
| | provirus genome | | | | | | | | | |
| X03475 | ribosomal protein L35a | V | — | — | — | — | — | K | — | — |
| X04979 | apolipoprotein E | — | — | V | K | — | — | K | — | — |
| X06554 | myelin-associated | — | V | — | — | — | — | — | — | — |
| | glycoprotein | | | | | | | | | |
| X06769 | FBJ osteosarcoma | — | — | V | — | K | V | K | K | — |
| | oncogene | | | | | | | | | |
| X06827 | hydroxymethylbilane | V | V | V | — | — | — | — | K | — |
| | synthase | | | | | | | | | |
| X06942 | v-raf murine sarcoma 3611 | — | — | V | — | — | — | — | — | — |
| | viral oncogene homolog | | | | | | | | | |
| X07286 | PKC, alpha | — | V | — | V | — | — | — | — | — |
| X07467 | glucose-6-phosphate | — | — | — | — | K | — | — | — | — |
| | dehydrogenase | | | | | | | | | |
| X07729 | enolase 2, gamma, neuronal | — | V | — | — | — | — | — | — | — |
| X13016 | Cd48 molecule | V | G | V | — | K | V | K | K | — |
| X13804 | neurofilament, heavy | — | G | — | — | — | — | — | — | — |
| | polypeptide | | | | | | | | | |
| X15013 | ribosomal protein L7a | V | — | — | — | — | V | — | — | G |
| X17184 | AMPAR 1 | — | V | — | — | V | K | — | — | K |
| X17621 | potassium voltage gated | — | V | — | — | V | — | — | — | — |
| | channel, shaker related | | | | | | | | | |
| | subfamily, member 6 | | | | | | | | | |
| X51992 | GABA A receptor, alpha 5 | — | — | — | K | — | — | K | G | — |
| X55812 | CB1 receptor | G | G | — | — | K | K | — | — | K |
| X56917 | IP3 3-kinase A | — | G | — | — | — | K | — | G | K |
| X57514 | GABA A receptor, gamma 1 | V | — | — | — | — | — | K | — | — |
| X58149 | Phosphoribulokinase, | — | — | — | — | — | — | — | G | — |
| | chloroplastic | | | | | | | | | |
| X61159 | glycine receptor, alpha 2 | — | G | — | — | K | — | K | — | — |
| X62085 | hypoxanthine | — | G | — | V | K | — | — | G | — |
| | phosphoribosyltransferase 1 | | | | | | | | | |
| X62295 | angiotensin II receptor, type | — | V | — | — | — | — | — | — | — |
| | 1a | | | | | | | | | |
| X62314 | somatostatin receptor 1 | — | — | — | K | — | — | — | G | — |
| X62840 | potassium voltage gated | V | — | G | — | — | — | K | — | — |
| | channel, Shaw-related | | | | | | | | | |
| | subfamily, member 1 | | | | | | | | | |
| X62841 | potassium voltage gated | — | — | G | — | — | — | — | — | G |
| | channel, Shaw-related | | | | | | | | | |
| | subfamily, member 4 | | | | | | | | | |
| X62952 | vimentin | V | V | G | — | — | — | K | K | G |
| X63143 | syndecan 3 | — | — | — | — | — | V | — | — | — |
| X63995 | solute carrier family 6 | — | — | — | — | V | — | — | — | K |
| | (neurotransmitter | | | | | | | | | |
| | transporter, serotonin), | | | | | | | | | |
| | member 4 | | | | | | | | | |
| X66842 | serotonin receptor 2B | — | V | — | — | — | — | — | — | — |
| X66870 | lamin A | V | V | — | V | V | V | G | G | — |
| X69903 | interleukin 4 receptor, alpha | — | — | — | — | V | — | — | — | — |
| X70521 | sodium channel, nonvoltage- | — | — | — | — | — | K | — | — | — |
| | gated, type I, alpha | | | | | | | | | |
| X70662 | potassium voltage-gated | — | — | — | — | — | — | — | G | — |
| | channel, shaker-related | | | | | | | | | |
| | subfamily, beta member 1 | | | | | | | | | |
| X73653 | glycogen synthase kinase 3 | — | V | — | — | — | — | — | — | — |
| | beta | | | | | | | | | |
| X73683 | similar to H3 histone, family | V | V | V | — | — | — | K | K | — |
| | 3B | | | | | | | | | |
| X74833 | cholinergic receptor, | — | V | — | — | — | — | — | — | — |
| | nicotinic, beta 1 (muscle) | | | | | | | | | |
| X76489 | CD9 molecule | V | — | — | V | — | — | K | — | — |
| X78606 | RAB28, member RAS | — | G | — | — | K | — | — | — | — |
| | oncogene family | | | | | | | | | |
| X79321 | microtubule-associated | — | G | — | — | K | — | — | — | — |
| | protein tau | | | | | | | | | |
| X83094 | heat shock transcription | G | — | — | — | — | K | — | — | — |
| | factor 1 | | | | | | | | | |
| X83580, | potassium inwardly-rectifying | G | G | — | — | K | K | G | G | K |
| X87635 | channel, subfamily J, | | | | | | | | | |
| | member 4 | | | | | | | | | |
| X83585 | potassium inwardly-rectifying | — | — | — | — | — | K | — | — | — |
| | channel, subfamily J, | | | | | | | | | |
| | member 10 | | | | | | | | | |
| X86789 | synuclein, gamma (breast | — | — | — | — | — | V | — | — | — |
| | cancer-specific protein 1) | | | | | | | | | |
| X91810 | signal transducer and | — | V | — | V | V | — | — | — | — |
| | activator of transcription 3 | | | | | | | | | |
| X92070 | purinergic receptor P2X, | — | — | — | — | V | — | — | G | — |
| | ligand-gated ion channel, 6 | | | | | | | | | |
| X95096 | Macrophage stimulating 1 | — | — | — | — | — | V | — | — | G |
| | (hepatocyte growth factor- | | | | | | | | | |
| | like) | | | | | | | | | |
| X95466 | spectrin repeat containing, | G | G | — | — | K | K | G | — | K |
| | nuclear envelope 1 | | | | | | | | | |
| X95579 | GABA receptor, rho 1 | — | V | — | — | V | K | — | — | — |
| X96488 | mitogen-activated protein | — | — | — | — | V | — | — | G | — |
| | kinase 12 | | | | | | | | | |
| X97121 | neurotensin receptor 2 | — | V | — | — | — | — | — | K | — |
| X97374 | prepronociceptin | — | — | — | — | — | — | G | — | — |
| X98564 | potassium channel, | — | — | — | — | — | K | — | — | — |
| | subfamily V, member 1 | | | | | | | | | |
| XM_001060 | eukaryotic translation | — | — | — | — | — | K | — | — | K |
| 756 | initiation factor 4 gamma, 1 | | | | | | | | | |
| XM_001066 | eukaryotic translation | V | — | — | — | — | — | — | — | — |
| 554 | initiation factor 4E family | | | | | | | | | |
| | member 1B | | | | | | | | | |
| XM_217346 | PTK7 protein tyrosine kinase | V | G | — | — | — | — | K | G | — |
| | 7 | | | | | | | | | |
| XM_217785 | Rattus norvegicus TATA box | — | V | — | — | — | — | — | — | — |
| | binding protein (Tbp), | | | | | | | | | |
| XM_224987 | secreted frizzled-related | V | — | V | — | — | — | — | — | — |
| | protein 1 | | | | | | | | | |
| XM_226051 | wingless-type MMTV | V | V | — | — | — | — | — | — | — |
| | integration site family, | | | | | | | | | |
| | member 8A | | | | | | | | | |
| XM_234422 | c-fos | V | V | V | V | — | V | — | — | — |
| XM_235454 | forkhead box H1 | — | V | — | — | V | — | — | K | — |
| XM_235639 | wingless-type MMTV | G | V | — | — | — | — | G | — | — |
| | integration site family, | | | | | | | | | |
| | member 1 | | | | | | | | | |
| XM_237295 | wingless-type MMTV | — | — | — | — | — | — | — | — | K |
| | integration site family, | | | | | | | | | |
| | member 6 | | | | | | | | | |
| XM_237296 | wingless-type MMTV | — | — | — | V | K | — | — | K | — |
| | integration site family, | | | | | | | | | |
| | member 10A | | | | | | | | | |
| XM_575489 | neurogenic differentiation 6 | — | G | — | — | — | — | — | G | — |
| Y11433 | pyrimidinergic receptor P2Y, | — | V | — | — | — | — | — | — | — |
| | G-protein coupled, 4 | | | | | | | | | |
| Y14635 | amiloride-sensitive cation | — | — | — | — | — | — | — | — | K |
| | channel 1, neuronal | | | | | | | | | |
| Y16563 | bassoon | — | — | — | — | V | — | — | G | — |
| Z11558 | glia maturation factor, beta | G | V | — | — | — | — | G | — | — |
| Z12152 | neurofilament, medium | — | V | — | — | V | K | — | — | — |
| | polypeptide | | | | | | | | | |
| Z24721 | SOD 3, extracellular | — | — | V | — | V | V | — | — | — |

### Example 2

### GLYX-13 Induces Rapid Antidepressant-Like Effects without Dissociative Side Effects

The present study examined GLYX-13 for its potential as a clinically relevant antidepressant using multiple rat models of depression, and tested for ketamine-like side effects in rats. The study also examined whether the antidepressant-like effects of GLYX-13 required AMPA glutamate receptor activation, and whether GLYX-13 could facilitate metaplasticity.

### Methods:

Behavioral Pharmacology: Male Sprague-Dawley (SD) rats (2-3 Months old) were given injections of GLYX-13 (1-56 mg/kg IV; 1-100 mg/kg SC; 0.1-10 μg MPFC), ketamine (10 mg/kg IV; 0.1-10 μg MPFC), fluoxetine positive control (three doses at 10 mg/kg SC) or sterile 0.9% saline vehicle, either 20-60 min or 24 hrs before Porsolt testing. Pretreatment with NBQX (10 mg/kg IP) was used to test the role of AMPAR in the antidepressant-like effect of GLYX-13 (3 mg/kg IV) in the Porsolt test. Antidepressant-like drug effects were measured by decrease in floating time in the Porsolt test, decreased feeding latency in a novel but not familiar environment for the novelty-induced hypophagia (NIH) test, and decreased number of escape failures in the learned helplessness (LH) test. Ketamine like abuse potential and reward was measured by ketamine-like responding in drug discrimination testing and time spent in the drug paired side in the conditioned place preference assay. Ketamine-like disruptions in sensory-motor gating were measured by decreased pre-pulse inhibition. Ketamine-like sedation was measured by decreases in open field locomotor activity and operant response rate in a drug discrimination study. Molecular Pharmacology: Adult male SD rats were dosed with GLYX-13 (3 mg/kg IV), ketamine (10 mg/kg IV) or saline vehicle and sacrificed 24 hrs post dosing. MPFC and hippocampal slices were prepared, and cell surface expressing proteins were cross-liked by biotinylation. Cell surface expression of GluR1 and NR2B were measured by Western blot. Electrophysiology: Hippocampal slices were prepared from adult male SD rats 24 hours after a single injection of GLYX-13 (3 mg/kg IV), ketamine (10 mg/kg IV) or vehicle. LTP at Schaffer collateral-CA1 synapses was measured in response to three submaximal bouts of high-frequency Schaffer collateral stimulation (2×100 Hz/800 ms). The percent contribution of NR2B and NR2A-containing NMDARs to pharmacologically isolated total NMDAR conductance were measured in Schaffer collateral-evoked EPSCs of CA1 pyramidal neurons by using the NR2B-selective NMDAR antagonist ifenprodil (10 μM), and the NR2A-NMDAR selective antagonist NVP-AM077 (100 nM).

### Results:

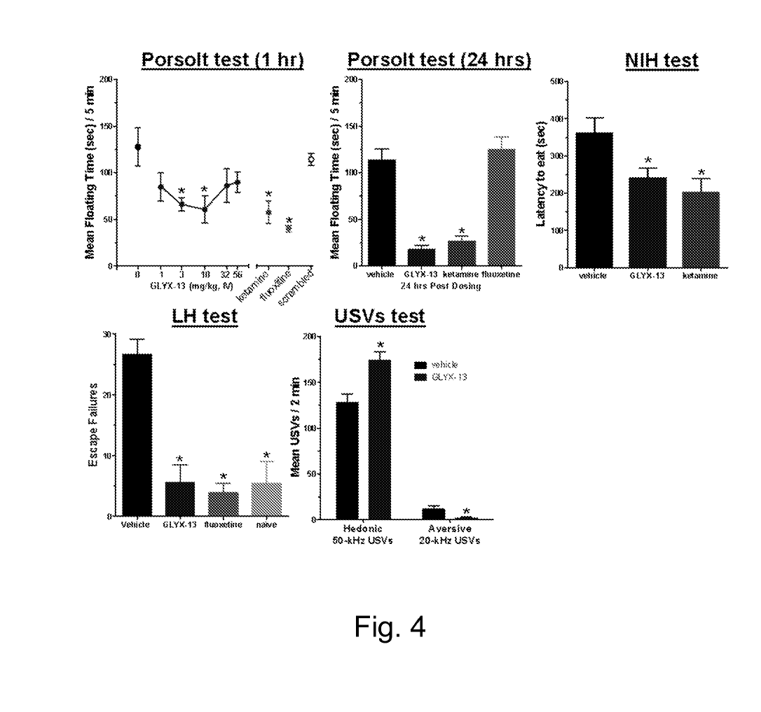

As shown in FIG. 4, GLYX-13 produces antidepressant-like effects in multiple rat models. The data were collected as described in the Methods and below.

Porsolt Test: 2-3 month old Sprague Dawley (SD) rats treated with a single dose of GLYX-13 (TPPT-NH3; 1-56 mg/kg, IV), scrambled GLYX-13 (PTTP-NH3; 3 mg/kg, IV), ketamine (10 mg/kg, IP), 3 doses of fluoxetine (20 mg/kg SC; 24, 5, and 1 hr before testing; (Detke et al., 1995)), or sterile saline vehicle (1 ml/kg, IV) 30-60 min before testing, or a single dose of GLYX-13 (3 mg/kg, IV), ketamine (10 mg/kg, IV) or 3 doses of fluoxetine (20 mg/kg SC; last dose 24 hrs before testing) or saline vehicle treated rats tested 24 hrs post dosing. NIH test: latency to eat in the novelty induced hypophagia (NIH) test in SD rats dosed with GLYX-13 (3 mg/kg, IV), ketamine (10 mg/kg, IV) or saline and tested 1 hr post dosing. LH test: escape failures in the footshock induced learned helplessness (LH) test in SD rats dosed with single dose of GLYX-13 (3 mg/kg IV; 24 hrs before testing), 3 doses Fluoxetine (20 mg/kg SC; last dose 1 hr before testing), or sterile saline vehicle (1 ml/kg IV; tail vein) 24 hrs before testing. Naïve control animals did not receive pre-shock or injection before LH testing. USVs test: Hedonic and Aversive USVs in adult SD rats receiving 2 min of heterospecific play (alternating blocks of 15 sec stimulation followed by 15 sec no stimulation). Data expressed as Mean (±SEM). N=7-21 per group. P<0.05 Fishers PLSD post hoc test vs. vehicle.

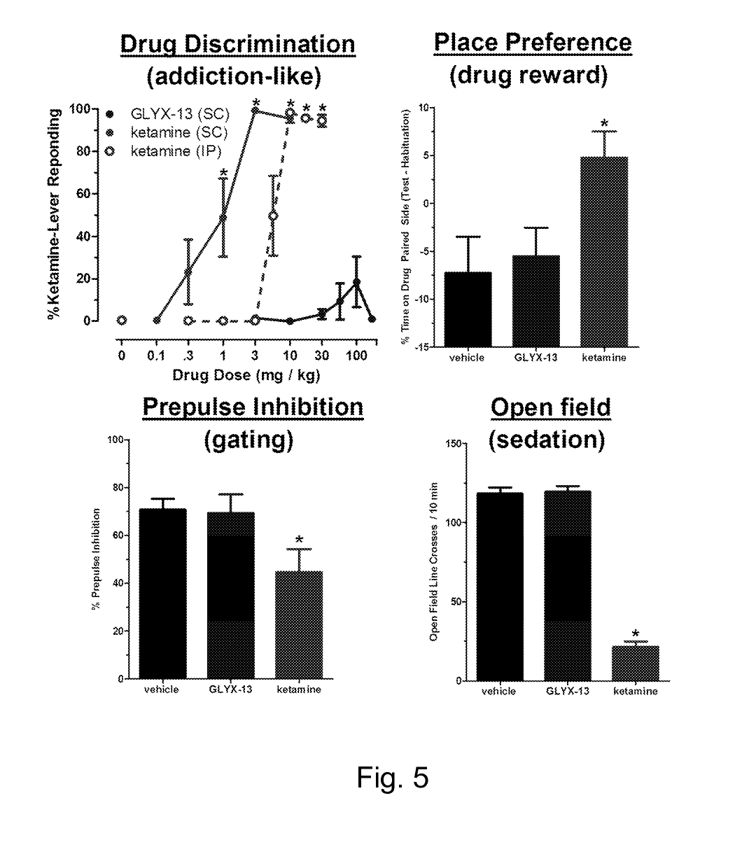

Results of the various tests presented in FIG. 5 demonstrate that GLYX-13 does not show ketamine-like addictive sensory motor gating, or sedative side effects. The data were collected as described in the Methods and below.

Drug discrimination: Percentage ketamine-lever responding and for different doses of ketamine (IP and SC) and GLYX-13 (SC) in SD rats trained to discriminate 10 mg/kg ketamine (Ket), IP, from saline (Sal). Values above Sal and Ket are the results of control tests conducted before testing each dose response curve. Place Preference: Ketamine (10 mg/kg IV) but not GLYX-13 (10 mg/kg IV) induced conditioned place preference as measured by % time in drug paired chamber. Prepulse Inhibition: Ketamine (10 mg/kg IP) but not GLYX-13 (10 mg/kg IV) decreased sensory-motor gating as measured by prepulse inhibition. Open field: A sedating dose of ketamine (10 mg/kg SC) but not GLYX-13 (10 mg/kg IV) reduced locomotor activity in the open field as measured by line crosses. N=8-11 per group. Data are expressed as Mean (±SEM). *P<0.05 Fishers PLSD post hoc test vs. vehicle.

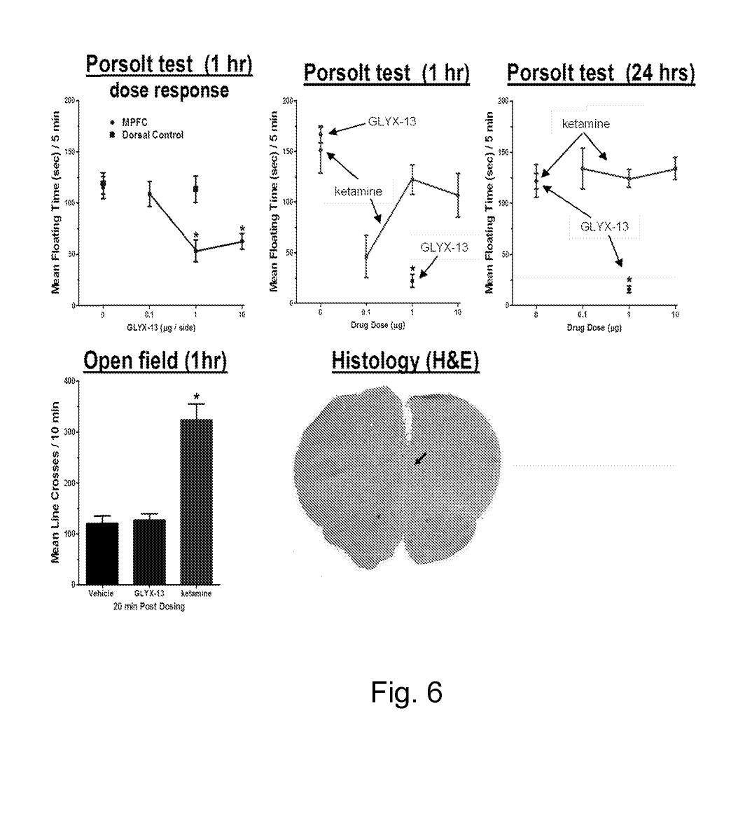

As indicated in FIG. 6, injection of GLYX-13 into the prefrontal cortex shows antidepressant-like effects in the Porsolt test. The data were collected as described below.

Mean (±SEM) time (sec) spent immobile in the Porsolt test in 2-3 month old male rats implanted with (a) medial prefrontal or motor cortex (dorsal control) cannulae and injected with GLYX-13 (0.1, 1, 10 μg/side) or sterile saline vehicle (0.5 μL/1 min) and tested 1 hr post dosing or rats given MPFC injections of ketamine (0.1, 1, 10 μg), GLYX-13 (1 μg), or saline and tested 20 min and 24 hrs post dosing. Animals received a 15 min training swim session one day before dosing. Mean (±SEM) line crosses in the open field 20 min following MPFC infusion of GLYX-13 (1 μg), ketamine (0.1 μg) or sterile saline vehicle. Given that 0.1 μg dose of ketamine increased locomotor activity, the Porsolt data for that dose were not included in the analysis given that increasing locomotor activity produces a false positive antidepressant-like response. A representative H&E stained section depicting MPFC cannulae placemen, arrow indicates injection site. N=5-10 per group. *P<0.05, Fisher PLSD vs. vehicle

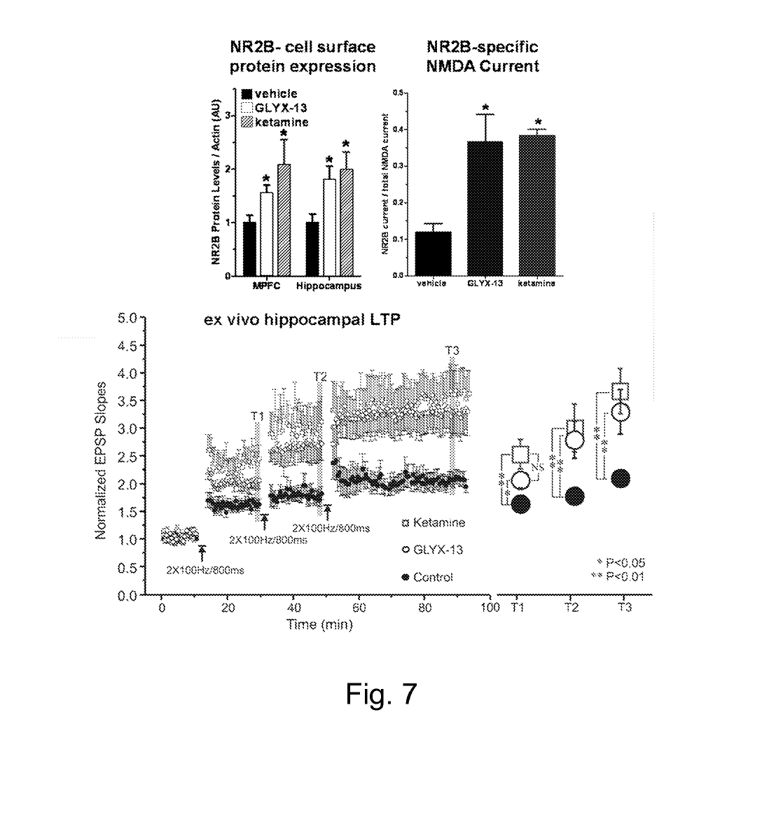

The data in FIG. 7 demonstrate that GLYX-13 induces NR2B-dependent synaptic plasticity. The data were collected as described in the Methods and below.

ex vivo cell surface protein levels: Biotinylated cell surface GluR1 protein levels in the medial prefrontal cortex (MPFC) or hippocampus as measured by western blot in SD rats treated with GLYX-13 (3 mg/kg IV) ketamine (10 mg/kg IV) or sterile saline vehicle 24 hours prior to sacrifice. ex vivo NMDAR current: NMDA receptor-dependent single shock-evoked EPSCs in the presence of the NR2B-selective NMDA receptor antagonist ifenprodil (10 μM), in CA1 pharmacological isolated NMDA current in rats that were dosed with GLYX-13 (3 mg/kg IV) ketamine (10 mg/kg IV) or sterile saline vehicle (IV) 24 hrs before ex-vivo NMDA current measurement. ex vivo LTP: GLYX-13 (3 mg/kg IV) or ketamine (10 mg/kg IV) 24 hrs post dosing enhances the magnitude of ex vivo long-term potentiation (LTP) of synaptic transmission at Schaffer collateral-CA1 synapses. Data are expressed as Mean (±SEM). N=5-11 per group. *P<0.05, **P<0.01 Fishers PLSD post hoc test vs. vehicle.

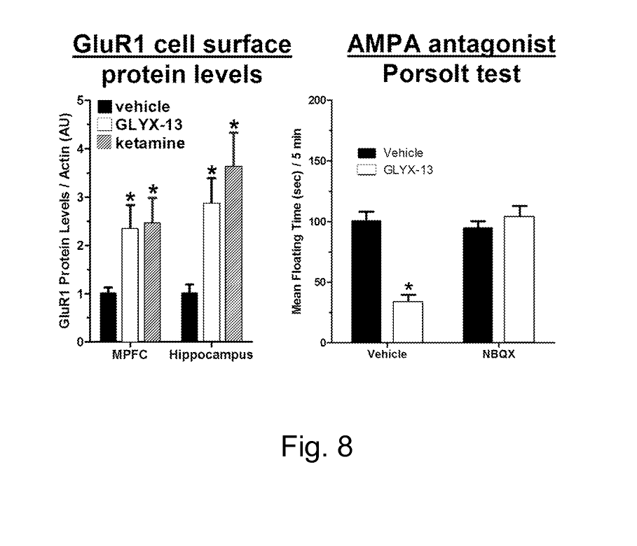

In FIG. 8, the data show that the antidepressant-like effects of GLYX-13 are synaptic-plasticity related. The data were collected as described in the Methods and below.

Ex vivo cell surface protein levels: Biotinylated cell surface GluR1 protein levels in the medial prefrontal cortex (MPFC) or hippocampus as measured by western blot in SD rats treated with GLYX-13 (3 mg/kg IV) or sterile saline vehicle 24 hours prior to sacrifice. AMPAR antagonism: Mean (±SEM) Floating time in the Porsolt test in animals pretreated with the AMPA receptor antagonist NBQX (10 mg/kg IP) before GLYX-13 (3 mg/kg IV) dosing and tested 1 hr post dosing.

### Summary

In total, the data show that (i) GLYX-13 produces a robust antidepressant-like effect without dissociative side effects; and (ii) GLYX-13 produce an antidepressant-like effect by facilitating synaptic plasticity in the MPFC.

### Example 3

### GLYX-13 Increases Ex Vivo [³H] MK-801 Binding, a Non-Competitive Antagonist of the NMDA Receptor, in the Rat Medial Prefrontal Cortex 1 Hour Post Dosing

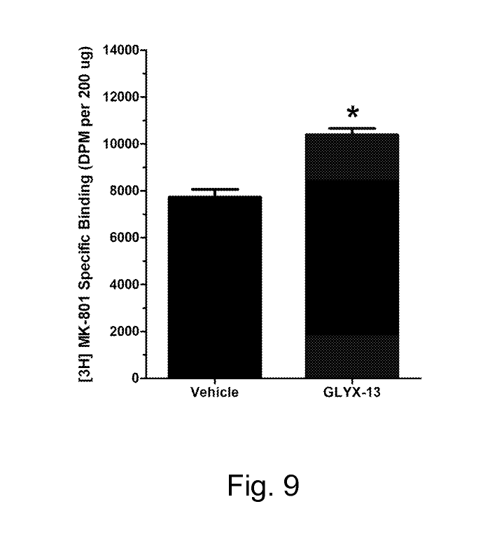

FIG. 9 shows that ex vivo [³H] MK-801 binding in the rat medial prefrontal cortex increases one hour after dosing with GLYX-13. The data were collected as described below.

Mean±SEM specific [³H] MK-801 binding (5 nM; 22.5 Ci/mmol) to well washed rat MPFC membranes (200 μg) in 2-3 month old Male SD rats treated with GLYX-13 (3 mg/kg IV) or sterile saline vehicle (1 ml/kg tail vein) and decapitated without anesthesia 1 hr post dosing, and brain rapidly removed (60 sec), frozen on dry ice, and stored at −80° C. until assay. [³H]MK-801 binding for was measured under equilibrium conditions (2 hrs) in the presence 1 mM glycine. Non-specific binding was were determined in the absence of any glycine ligand and in the presence of 30 μM 5,7 DCKA. Maximal stimulation was measured in the presence of 1 mM glycine. 50 μM glutamate was present in all reactions. n=5-6 per group. *P<0.05 vs. respective vehicle.

### Example 4

### Rapid Antidepressant Effects of GLYX-13 May be Mediated by an E-LTP-Like Mechanism

To examine the rapid-acting effects of GLYX-13, the biochemical processes that underlie the induction of early stage long term potentiation (E-LTP) were studied.

Without wishing to be bound by any theory, E-LTP is dependent upon the persistent activation of protein kinases, including Ca²⁺/calmodulin-dependent protein kinase (CAMKII), protein kinase C (PKC), and casein kinase II (CK2). GLYX-13 (3 mg/kg, IV), or vehicle, were administered to adult (2-3 months old) male Sprague-Dawley rats, and medial prefrontal cortex samples were collected at 15, 30, 60, and 120 min post-dosing (n=7-9 per group). Total cellular proteins were subjected to 7.5% SDS-PAGE and probed with antibodies directed against GluN2B (4207S, Cell Signaling, MA), pS-1303 GluN2B (Millipore, Mass.), or pS-1480 GluN2B (ab73014, Abcam, Mass.). Enhanced chemiluminescence was used to quantitate individual bands. CK2 activity was measured by phosphorylation of a CK2 substrate peptide using the transfer of the gamma-phosphate of [gamma-³²P]-ATP (Millipore, Mass.). Total protein (7.5 micrograms) was incubated with CK2 substrate peptide for 10 min in the presence of 0.1 microliters of stock [gamma-³²P]-ATP (100 nCi/reaction).

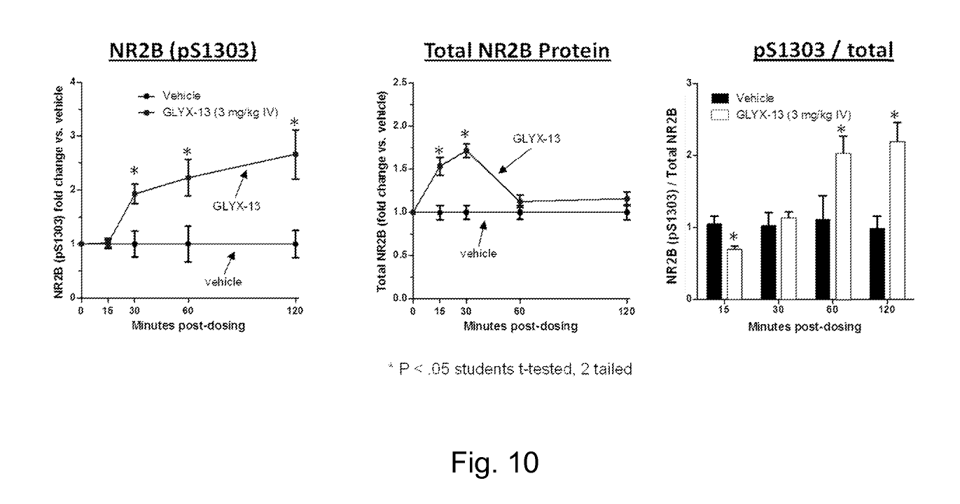

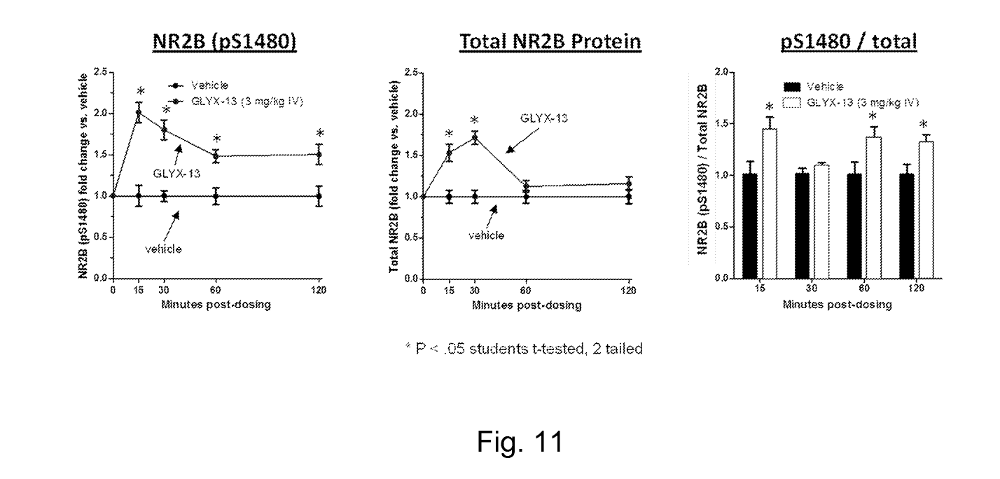

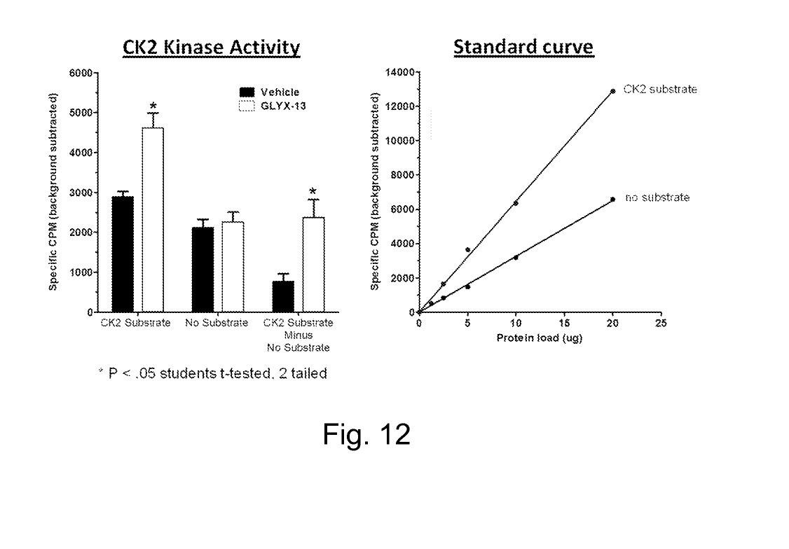

GLYX-13 led to a significant increase in total GluN2B protein within 15 min (1.53 fold vs. vehicle, P<0.05) of administration that peaked at 30 min (1.71 fold, P<0.05) and returned to control levels by 60 min (60 min, 1.13 fold, P >0.05; 120 min, 1.16 fold, P >0.05) (FIGS. 10 and 11). CAMKII/PKC-mediated serine-1303 phosphorylation of GluN2B levels were increased at the 30 min (1.93 fold, P<0.05), 60 min (2.23 fold, P<0.05), and 120 min (2.67 fold, P<0.05) timepoints but did not change at the 15 min timepoint (1.02 fold, P >0.05) (FIG. 10). CK2-mediated serine-1480 phosphorylation of GluN2B levels peaked within 15 min (2.01 fold, P<0.05) and remained significantly elevated up to 120 min (30 min, 1.80 fold, P<0.05; 60 min, 1.48 fold, P<0.05; 120 min 1.50 fold, P<. 05) after administration of GLYX-13 (FIG. 11). A significant increase in CK-2 specific activity was also observed at 15 min (3.08 fold, P<0.05) (FIG. 12). CK2 and CAMKII activity has been shown to be rapidly increased at the onset of LTP (Charriaut-Marlangue et al., 1991, PNAS, 88, 10232; Fukunaga et al., 1993, JBC, 268, 7863). This observation, along with the results reported here, suggest that the rapid onset of the antidepressant effect of GLYX-13 is mediated, at least in part, by the same mechanisms that regulate E-LTP.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, parameters, descriptive features and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

### INCORPORATION BY REFERENCE

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

What is claimed is:

1. A method for identifying a candidate compound suitable for treatment of depression, comprising:
exposing a cell to a potential compound in a culture medium, or administering a potential compound to an animal;
retrieving a sample from the cell and/or culture medium, or from brain or neural tissue of the animal, at one or more predetermined time points;
analyzing the sample for increased expression levels of Wnt1, and
identifying the candidate compound as suitable for treatment of depression based on the increased expression level of Wnt1.

2. A method for identifying a candidate compound suitable for treatment of depression, comprising:
exposing a cell to a potential compound in a culture medium, or administering a potential compound to an animal;
retrieving a sample from the cell and/or culture medium, or from brain or neural tissue of the animal, at one or more predetermined time points;
analyzing the sample for increased expression levels of at least one of the genes listed in Table 1 or 2 indicated with a G, or decreased expression levels or at least one of the genes listed in Table 1 or 2 indicated with a K, and
identifying the compound as suitable for treatment of depression based on the increased expression level or decreased expression level.

3. The method of claim 2, wherein the sample has a gene expression pattern as provided in Table 1 or 2 with the indication “G” and the identifying is based on increased expression of those genes.

4. The method of any one of claims 1-3, further comprising analyzing the candidate compound for NDMA subunit NR2B synaptic plasticity.

5. A method for identifying a compound suitable for treatment of depression, comprising:
exposing a cell to a potential compound in a culture medium, or administering a potential compound to an animal;
retrieving a sample from the cell and/or culture medium, or from brain or neural tissue of the animal, at one or more predetermined time points;
analyzing the sample for NMDA receptor NR2B subunit plasticity, and
identifying the compound as suitable for treatment of depression based on inducing the NR2B plasticity.

6. The method of claim 5, wherein a candidate compound suitable for treating depression significantly induces NR2B dependent synaptic plasticity as compared to ketamine.

7. The method of any one of claims 1-6, wherein the tissue is medial prefrontal cortex.

8. The method of any one of claims 1-7, wherein the animal is a rodent or human, and the cell is a human or rodent cell.

9. The method of any one of claims 1-7, wherein the compound modulates the NMDA receptor.

10. The method of any one of claims 1-8, wherein the compound suitable for treating depression has fewer side effects as compared to ketamine.

11. The method of claim 10, wherein the compound does not have substantial addictive sensory motor grating and/or sedative effect.

12. The method of any one of claims 1-11, wherein the cell is a eukaryotic cell.

13. The method of any one of claims 1-12, further comprising selecting the candidate compound from a library of compounds.

14. A method of identifying a therapeutic compound capable of treating depression in a patient, comprising selecting a compound that significantly induces NR2B dependent synaptic plasticity.
